(19) United States
(12) Patent Application Publication
(10) Pub. No.: US 2016/0331395 A1
(43) Pub. Date: Nov. 17, 2016

(54) ACCESS SYSTEM FOR LAPAROSCOPIC SURGERY

(71) Applicant: KATYA SURGICAL LTD., Tel-Aviv (IL)

(72) Inventor: Igor IGOV, Lod (IL)

(21) Appl. No.: 15/112,315

(22) PCT Filed: Jan. 18, 2015
(86) PCT No.: PCT/IL2015/050055

§ 371 Date: Jul. 18, 2016

Related U.S. Application Data
(60) Provisional application No. 61/929,029, filed on Jan. 18, 2014.
(60) Provisional application No. 62/000,678, filed on May 20, 2014.

(51) Int. Cl. A61B 17/32; A61B 18/14; A61B 18/08; A61B 17/3201; A61B 17/3209

(52) U.S. Cl. CPC A61B 17/320016; A61B 17/3201; A61B 17/32093; A61B 18/085; A61B 18/1445; A61B 2018/00595

(57) ABSTRACT

Multiple tools may be inserted into a body and/or controlled using a laparoscopic access system. For example the lap-aroscopic access system, may include a rigid tube adapted for insertion into a human body and long enough for laparoscopic use; a handle including a manipulator coupled to the tube; and a tool body including a tool head, slidably received in the rigid tube and long enough to extend from the manipulator with the tool head extending out a distal opening of the rigid tube and wherein the tool body mechanically couples force from the manipulator to the tool head.

### RELATED APPLICATION/S

This application claims the benefit of priority under 35 USC §119(e) of U.S. Provisional Patent Applications: 61/929,029 filed 18 Jan. 2014, 62/000,678 filed 20 May 2014, the contents of which are incorporated hereto by reference in their entirety.

### FIELD AND BACKGROUND OF THE INVENTION

The present invention, in some embodiments thereof, relates to a system for controlling tools in a cavity of a living creature and, more particularly, but not exclusively, to a system for changing tools in a laparoscopic access system.

Background an includes U.S. patent application Ser. No. 6,077,290; U.S. patent application Ser. No. 7,025,775; U.S. patent application Ser. No. 5,755,713; German patent application no. 102009025013; U.S. patent application Ser. No. 7,297,142; German patent application no. 10149421; U.S. Patent Application Publication No. 2011/0112517, U.S. Design Pat. No. D404484; U.S. patent application Ser. No. 5,224,930; U.S. patent application Ser. No. 4,924,851; U.S. patent application Ser. No. 7,455,679; and International Patent Application Publication No. 2014113428.

### SUMMARY OF THE INVENTION

According to an aspect of some embodiments of the invention, there in provided a laparoscopic tool system, comprising: (a) a rigid tube adapted for insertion into a human body and long enough for laparoscopic use; (b) a handle coupled to the tube; and (c) a tool body attachable to a manual manipulator and including a tool head, slidably received in the rigid tube and long enough to extend from the manipulator with the tool head extending out a distal side of the rigid tube and wherein the tool body mechanically couples force from the manipulator to the tool head, wherein the system, when the rigid tube is fully inserted into a body, rigidly extends less than 60% a length of the rigid tube out of the body.

According to some embodiments of the invention, the tool head is passable from a proximal opening to a distal opening of the rigid tube.

According to some embodiments of the invention, the tool body is slidable to extend at least half its length proximally from the handle.

According to some embodiment of the invention, the rigid tube is adapted to have a length of at least 15 cm inserted into a body.

According to some embodiments of the invention, the handle is adapted to extend at most 20 cm from a body, when the rigid tube is maximally inserted into a body.

According to some embodiments of the invention, only the rigid tube is insertable into the body through a laparoscopic opening with at most one tool body and one tool head therein.

According to some embodiments of the invention, the tool body is axially coupled to the rigid tube and wherein the manipulator is axially coupled and transaxially coupled to the tool body.

According to some embodiments of the invention, the tool body comprises at least one elongate flexible tension coupling element.

According to some embodiments of the invention, the tool body comprises two elongate rigid elements, both engaged by the manipulator.

According to some embodiments of the invention, the tool body comprises an outer tube and an inner shaft slidebly received therein, both engaged by the manipulator.

According to some embodiments of the invention, mechanically coupling comprises providing simultaneously a pushing force and a pulling force to the tool head.

According to some embodiments of the invention, the tool head is separable from the tool body at art attachment area closer to a distal end of the tool body than to a proximal end thereof.

According to some embodiments of the invention, the rigid tube envelopes the attachment area when the tool body engages the manipulator and the tool head extends from the rigid tube distal opening.

According to some embodiments of the invention, the system comprises a magazine holding a plurality of tool heads suitable for attachment to the tool body.

According to some embodiments of the invention, the magazine is configured to selectably extend away from the handle.

According to some embodiments of the invention, the attachment area comprises a sliding attachment, by trans-axial sliding of the tool head.

According to some embodiments of the invention, the handle defines an aperture for transaxially moving the tool head during attachment thereof.

According to some embodiments of the invention, the system according to my of claims comprises a pusher to transaxially push the tool head away from the tool body.

According to some embodiments of the invention, the system according to any of claims comprises a grasper to transaxially pull the tool head away from the tool body.

According to some embodiments of the invention, the tool body includes at least one geometrical element near a distal end thereof to which is configured to interact with a matching geometrical element in the handle and prevent over retraction of the tool body.

According to some embodiments of the invention, the tool head is permanently attached to the tool body and wherein the tool body and the tool head are slidably removable from the handle by proximal retraction thereof through an aperture n a proximal side of the handle.

According to some embodiments of the invention, the rigid tube includes at least one narrowing at or near a distal end thereof, which is sized and positioned to prevent over insertion of the tool body.

According to some embodiments of the invention, the rigid tube includes at least one changeable narrowing at or near a proximal end thereof, which is sized and positioned to prevent unintended retraction of the tool body.

According to some embodiments of the invention, the tool body is attachable and detachable from the manipulator without applying more than 5 Kg of force.

According to some embodiments of the invention, the tool body is attachable and detachable from the manipulator, while the system is inserted in the body, without moving a distal end of the rigid tube more than 3 cm in any direction.

According to some embodiments of the invention, the tool body is both mechanically and electrically coupled to the manipulator.

According to some embodiments of the invention, the manipulator engages a narrowing in the tool body.

According to some embodiments of the invention, the rigid tube has an average wall thickness of less than 0.5 mm.

According to some embodiments of the invention, the rigid tube has an inner diameter within 10% of an outer diameter of the tool body.

According to some embodiments of the invention, the rigid tube has an inner diameter closer enough to an outer diameter of the tool body to substantially prevent air and blood leaks therethrough.

According to some embodiments of the invention, the rigid tube has an inner diameter and the tool body has an outer diameter of the tool body, both of which are uniform over at least 90% of their lengths.

According to some embodiments of the invention, the system comprises a seal between the rigid tube and the tool body.

According to some embodiments of the invention, the seal comprises a valve operative in maintain a seal also when the tool body is retracted away from the rigid tube distal opening.

According to some embodiments of the invention, the system comprises a flexible filament tool passed between the rigid tube and the tool body.

According to some embodiments of the invention, the flexible filament tool comprises two ends, a first end engaged by the tool head and a second end passing between the rigid tube and the tool body, such that pulling on the second end relative can close a loop.

According to some embodiments of the invention, the flexible filament includes at least on material selected from the group consisting of a string, a wire, single filament nylon, and a braided strand.

According to an aspect of some embodiments of the invention, there is provided a system according to any of the preceding claims and provided in kit for with a plurality of tool heads.

According to an aspect of some embodiments of the invention, there is provided a magazine comprising plurality of tool heads configured to be mounted on a system according to any of claims 12-20.

According to an aspect of some embodiments of the invention, there is provided a method of laparoscopic surgery, comprising: (a) inserting tool head into the body at the end of a tool body coupled to a tool handle, to a location in the body; (b) retracting the tool head and the tool body out of the body and at least partially out of the handle; (c) advancing a different tool head into the body to the location; and (d) manipulating the tool head using a manipulator coupled to the tool handle.

According to some embodiments of the invention, the inserting and advancing use a same rigid tube as a guide which reaches to the location.

According to some embodiments of the invention, the retracting comprises completely separating the tool body from the handle.

According to some embodiments of the invention, the retracting comprises separating the tool head from the tool body.

According to some embodiments of the invention, the method comprises replacing the tool head by other than a physician who performs the manipulating.

According to some embodiments of the invention, the method comprises initially inserting the tool body into an abdomen without a port.

According to some embodiments of the invention, the method comprises forming an incision in the abdomen using a tool at an end of the tool body.

According to some embodiments of the invention, the tool is a dedicated incision maker.

According to some embodiments of the invention, the tool comprises an expandable tissue dissector.

According to an aspect of some embodiments of the invention, there is provided a cutting laparoscopic tool, comprising: a tool body having a slot formed in a distal end portion thereof; and a blade slidably received in the slot and mounted on a rigid elongate extender and having an outer diameter within 15% of an outer diameter of the tool body.

According to some embodiments of the invention, the slot is formed in a distal end of a tool head forming a distal end of the tool body, the tool head configured to open and close.

According to some embodiments of the invention, the slot is formed in a proximal end of a tool head forming a distal end of the tool body and the tool head does not allow the blade to extend past the tool head.

According to some embodiments of the invention, the blade is configured for forming on abdominal incision for advancement of the tool body therethrough.

According to some embodiments of the invention, the blade is configured for cutting tissue held by the tool head, within the tool head.

According to an aspect of some embodiments of the invention, there is provided a laparoscopic hook, comprising: a tool body having an outer diameter; at least one extendible element; and a manipulator configured to selectively rotate the extendible element to extend 90 degrees or less from the tool body and be usable to hook and retract tissue therewith.

According to some embodiment of the invention, the manipulator comprises an overtube and wherein the extendible element is not straight, such that advancing the overtube engages a first part of the extendible element, so that a different part of the extendible element is thereby rotates out of a path of the tool body.

According to an aspect of some embodiments of the invention, there is provided a laparoscopic cutter, comprising: (a) a tool body including a cutaway section; (b) a blade slidably received within the tool body and slidable across a gap of the cutaway section.

According to some embodiments of the invention, the gap is wider at its base than at a top opening thereof.

According to some embodiments of the invention, the blade is longer at a side near the top than near the base. Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are block diagrams of a laparoscopic access system with a retainer reversibly locking a tool to an access tube in accordance with an embodiment of the present invention;

FIG. 2 is a block diagram of a laparoscopic access system with a retainer reversibly locking a control rod to an access tube in accordance with an embodiment of the present invention;

FIG. 3 is a flow chart illustrating a method of deploying multiple tools with a single laparoscopic access system in accordance with an embodiment of the present invention;

FIGS. 4A-B, 5, 6A-B and 7 illustrate a method of deploying multiple tools with a single laparoscopic access system in accordance with an embodiment of the present invention;

FIGS. 8A-H illustrate a handle and control lever for an a laparoscopic access system with two side by side control rods in accordance with an embodiment of the present invention;

FIGS. 9A-D illustrate a distal retainer for a tool of a laparoscopic access system in accordance with an embodiment of the present invention;

FIGS. 10A-C illustrate a handle and control lever for a laparoscopic access system with inner and outer control rods in accordance with an embodiment of the present invention;

FIGS. 11A-C illustrate a guillotine cutter laparoscopic tool in accordance with an embodiment of the present invention;

FIGS. 12A-D illustrate a dissector laparoscopic tool in accordance with an embodiment of the present invention;

FIGS. 13A-C illustrate a dissector laparoscopic tool employing a balloon in accordance with an embodiment of the present invention;

FIGS. 14A-D illustrate a hook laparoscopic tool in accordance with an embodiment of the prevent invention:

FIGS. 15A-B illustrate a cauterizer laparoscopic tool in accordance with an embodiment of the present invention:

FIG. 15C illustrates a balloon dissector laparoscopic tool in accordance with an embodiment of the present invention;

FIGS. 16A-B illustrate a string activated hook laparoscopic tool in accordance with an embodiment of the present invention;

FIGS. 17A-B illustrate a string retracted hook laparoscopic tool in accordance with an embodiment of the present invention;

FIGS. 18A-H illustrate grasper and/or dissector laparoscopic tool with a cutter and/or puncture tool in accordance with an embodiment of the present invention;

FIGS. 19A-E illustrate use of a grasper and/or dissector laparoscopic tool with a cutter to remove a gall bladder in accordance with an embodiment of the present invention;

FIGS. 20A-F illustrate use of a grasper and/or dissector laparoscopic tool with a cutter to cut an intestinal adherence in accordance with an embodiment of the present invention;

FIG. 21 illustrates use of a grasper and/or dissector laparoscopic tool with a cutter and/or cauterization to cut a blood vessel in accordance with an embodiment of the present invention;

FIGS. 22A-C illustrate use of a grasper and/or dissector laparoscopic tool to tie off an organ in accordance with an embodiment of the present invention;

FIGS. 23A-F, 24A-C and 25A-B illustrate applying a clip in accordance with an embodiment of the present invention;

FIGS. 26A-D illustrate a pistol style handle for a laparoscopic access system in accordance with an embodiment of the present invention;

FIGS. 27A-B illustrate a revolver style handle for a laparoscopic access system in accordance with an embodiment of the present invention; and

FIG. 28 illustrates an automatic magazine style handle for a laparoscopic access system in accordance with an embodiment of the present invention.

### DESCRIPTION OF SPECIFIC EMBODIMENTS OF THE INVENTION

The present invention, in some embodiments thereof, relates to a system for controlling tools in a cavity of a living creature and, more particularly, but not exclusively, to a system for changing tools in a laparoscopic access system.

### Overview

A broad aspect of some embodiments of the invention relates to laparoscopic surgery where a tool head is retracted out of the body and replaced. In an exemplary embodiment of the invention, the tool head is mounted on or integrally connected in a tool body which is retracted out of the body with the tool head and which serves to convey mechanical forces from an out-of-body manipulator/handle to the tool head.

An aspect of some embodiments of the invention relates to using an axially extending guide tube, adapted to be inserted into a laparoscopic incision and/or port and which guides a replaceable tool head to a work location in the body and which allows replacement of the head without substantial movement of the guide tube. In an exemplary embodiment of the invention, the distal end of the guide tube moves less than 10 cm, 5 cm, 3 cm, 2 cm or may not even move at all when the tool head is replaced. In an exemplary embodiment of the invention, the guide tube is rigid, for example, able to resist bending at one or more of the combinations of more than 30 mm. 15 mm, 10 mm, 5 mm or less under a lateral force at a distal end of 20 Kg, 10 Kg, 5 kg, 2 Kg, 1 Kg, 500 grams or intermediate amounts, while being held at a proximal end thereof. In some embodiments, such rigidity is provided by the cooperation of the tube and the tool body. Optionally the tube may be partially flexible. For example a distal portion of the tube may be flexible. For example the flexible portion may be less than 2 cm and/or between 2 to 5 cm and/or between 5 to 10 cm.

In an exemplary embodiment of the invention, the rigid tube is adapted to be inserted at least 10 cm, 20 cm, 30 cm, 40 cm or intermediate or greater depths into the body (e.g., until stopped by a widening thereon and/or the handle).

It is a particular feature of some embodiments of the invention that the rigid tube is not used to convey mechanical forces from a handle to the tool head and/or does not act against the tool head other than to maintain the tool head in a correct position and/or orientation. Optionally, any such force is applied to the tool body, not the tool head. In some embodiments, the rigid tube is used for applying axial forces to the tool body and/or transaxial forces, for example, to assist in manipulating the tool head, for example, opening and closing a grasper thereof.

In an exemplary embodiment of the invention, the tool body is rigid and is slidably received in said guide tube. In some embodiments, the tool body includes at least one flexible or otherwise deformable part, however, the guide tube acts to prevent deformation of the tool body at that part. In some embodiments, the deformable part is a tool head attachment area, which is deformable by trans-axial sliding and/or rotating of the tool head away from the tool body, so that the tool body can be separated from the tool head.

It is a particular feature of some embodiments of the invention that during use, the handle of the access system does not need to extend more than a standard extension. This may prevent the tool from interfering with work flow in the surgical arena. In an exemplary embodiment of the invention, the handle does not extend more than 70%, 50%, 30%, 20% of a length of the guide tube away from the body. For example, the handle may extend (when the system is fully inserted), less than 30 cm, 20 cm, 15 cm 10 cm or intermediate amounts.

In some exemplary embodiments of the invention, the tool body comprises at least two parts which can move one relative to the other, for example, to deliver mechanical movement to the tool head. In an exemplary embodiment of the invention, one part is within or side by side with the other. In an exemplary embodiment of the invention, the guide tube does not serve to convey forces for activating the tool head.

In some exemplary embodiments of the invention, the laparoscopic system includes a handle on which said rigid tube is mounted and/or integral with and the handle includes a manipulator, for example, a scissors grip. In an exemplary embodiment of the invention, both parts of the tool body are mechanically coupled to the manipulator. Optionally, electrical coupling of the tool head to the handle is provided via the tool body.

In some exemplary embodiments of the invention, a separate engagement is provided to prevent relative movement of said tool body relative to said rigid tube. In an exemplary embodiment of the invention, one or more geometries defined on the tool body and the rigid tube prevent over insertion of the tool body, for example, a narrowing at an end of the rigid tube. Optionally, the rigid tube includes a guide element, for example, a rail, or otherwise has a non-circular cross-section (e.g. with a cooperating/matching tool body cross-section), so as to control an orientation of the tool body relative to the rigid tube.

In an exemplary embodiment of the invention, the manipulator is designed for easy attachment and detachment from the tool body. This may be useful to allow retraction of the tool body from said handle without moving the rigid tube too much. Optionally or alternatively, the handle is designed for easy locking in place of the tool body, for example, to prevent excessive movement of the distal end of the rigid tube. In an exemplary embodiment of the invention, such replacements are performed by a person other than a surgeon using the tool for manipulation, for example, a nurse. In some exemplary embodiments of the invention, the maximal force required for such detachment and/or attachment is less than 10 Kg, 5 Kg, 3 Kg, 1 Kg, 500 grams, 250 grams and/or smaller or intermediate forces. Optionally, the forces can be applied by one hand, while another hand holds the system in place.

In some embodiments of the invention, the tool head is permanently affixed to the tool body and head replacement is by retracting the tool body out of said handle. Optionally, a bag is affixed to the handle to receive said retracted tool head and/or contain a replacement tool body/head. Optionally, the laparoscopic system is packaged with a plurality of tool body/head assemblies.

In some exemplary embodiments of the invention, the tool head is detachable from the tool body. In some exemplary embodiments of the invention, the tool body is retracted out of the body and mostly out of the handle (e.g., possibly exposed to the operating room), so that the tool head is outside the body. In an exemplary embodiment of the invention, the handle includes a geometry which cooperates with a geometry on the tool body to prevent unintentional over retraction of the tool body. For example, the handle may include a protrusion and the tool body a groove over most of its length, up to a distal point when the groove stops and thereby prevents over retraction. Optionally, the handle includes an aperture through which the tool head can be accessed. Optionally, the handle includes a button or lever which applies lateral force on the tool head to cause it to exit through the aperture. In some embodiments a magazine with multiple tool heads is provided and which can be selectively mounted on the tool body. Optionally, the magazine can be popped-out when a tool head is to be replaced for visual selection of a tool by a tool replacing person. Optionally or alternatively, the magazine includes a grasper for pulling said tool head out of said handle aperture. Optionally the magazine can be removed and the handle can be loaded and/or unloaded with one tool at a time. Optionally switching tools in the magazine may be mechanical and/or by means of a powered actuator (for example an electrical actuator).

In an exemplary embodiment of the invention, the rigid tube serves to prevent disassociation of the tool head and the tool body by enclosing the attachment part. Optionally, the tool head is less than 20 cm, 15 cm, 10 cm or 5 cm long. Optionally or alternatively, the tool head is at least 3 cm, 5 cm and/or 10 cm long. Optionally, the attachment area is between 1 and 20 cm long, optionally, between 12 and 5 cm long. In an exemplary embodiment of the invention, the attachment is a sliding attachment, with at least one and optionally two interference elements (e.g., protrusions on one of tool head and tool body matching a recess on the other, which provide axial interference to prevent detachment of the tool head from the tool body. Optionally, non-axial movements are prevented, at least in part by the rigid tube. Optionally or alternatively, the interferences are dovetailed (e.g., wider at end than at base, and/or inclined), so that only relative movement in one direction is supported.

In some embodiments, the portion of the system that is inserted into the body has space for only one tool at a time. For example the largest inner diameter of the portion of the system in the body may range between 1 and 1.1 times the outer diameter of a tool and/or between 1 and 1.5 times the outer diameter of the tool and/or between 1 and 2 times the outer diameter of the tool.

In some embodiments, the distal end of the tool may be retracted to less than 10 cm from the proximal end of the tube and/or handle and/or less than 20 cm from the proximal end of the tube.

In some exemplary embodiments of the invention, the spacing between the tool body and the guide tube is tight enough to reduce or prevent leakage of air and/or blood therebetween. In an exemplary embodiment of the invention, the difference between the tube inner diameter d the tool body outer diameter is less than 20%, 10%, 5%, 3% or intermediate percentages of the tube inner diameter.

Optionally or alternatively, a seal, for example, a rubber ring, is provided in the guide tube and/or on the tool body. Optionally or alternatively, a valve is provided in the access system, for example, in the handle or in the rigid tube, which prevent fluid flow out of the body even when no tool body is inverted.

In some exemplary embodiments of the invention, the handle can be attached to different length and/or diameter tubes, for example, for different needs. Optionally, a standard size manipulator attachment is provided for tool bodies with a range of diameters. For example, different diameter tool bodies may have same size and/or shape attachment parts for attachment to a manipulator.

In an exemplary embodiment of the invention, the tube attachment is by screwing thereof into a socket in the handle and/or using a clamp on the handle to engage the rigid tube.

In some embodiment of the invention the handle (or a widening thereof and/or thereon) acts as a stop to prevent over insertion of the handle and rigid tube into the body. Optionally, however, the rigid tube can be partially retracted to control the position of the tool head in the body.

In some embodiments tube is connected on a proximal portion thereof to a handle. The handle optionally includes a handgrip. The handle is optionally coupled to tube. For example, handle may be rigidly connected to tube for example the handle may include a tube connector. Additionally or alternatively, a tube and handle may be formed together and/or be formed of a single material. Handgrip is optionally used to control the position of tube. Optionally, tube is rigid and/or partially rigid. For example handgrip may be used to control the position of the distal portion of tube from a proximal portion thereof. Optionally, the lever is connected via a lever connector to the shaft. Optionally, lever is connected via a handle connector to the handle and/or the tube. For example the handle connector may include a pivot. Optionally, the handgrip and/or the lever are located on proximal portion of tube. Optionally, the handgrip and/or lever can both be controlled with one hand. For example, the handgrip and/or lever and/or handle connector may have a form like the handle of a pair of scissors.

In an exemplary embodiment of the invention, the rigid tube with the head extending therefrom is between 20 and 60 cm in length. Optionally or alternatively, the outer diameter of the rigid tube is between 1 and 15 mm, for example, between 2 and 6 mm. Optionally, the diameter is such that the tube can be inserted into the body without a port or with a microport and the incision can be closed with no or one or two stitches.

In an exemplary embodiment of the invention, the rigid tube has a wall thickness of less than 1 mm, 0.5 mm, 0.3 mm, 0.1 mm intermediate thicknesses.

In an exemplary embodiment of the invention, the rigid tube and/or the tool body have uniform geometries, for example having uniform cross-sections and/or thicknesses over at least 70%, 80%, 90% and/or 95% of one or both of their lengths. As noted herein, the cross-sections may be matching or otherwise cooperating. Also as noted, the cross-sections need not be circular, which may assist, for example, in orientation control.

An aspect of some embodiments of the invention relates to using a tool head which has substantially the same (or smaller) diameter as the tool body and can be slidebly received in the guide tube.

An aspect of some embodiments of the invention relates to self-penetrating laparoscopic tools, for example, an access system as described above. In an exemplary embodiment of the invention, the tool includes a selectively advanceable blade at its distal tip which can be advanced to allow forming an opening in an abdominal wall. In an exemplary embodiment of the invention, the blade advances through a tool head with a different function, for example, grasping or cutting.

An aspect of some embodiments of the invention relates to controlled cutting. In an exemplary embodiment of the invention, a tool head defines a gap into which tissue can be received and a blade is slidably advanced across the gap to cut the tissue. Optionally, the gap is permanent. Optionally, the gap is wider inside than at an opening thereof, to optionally prevent tissue escape. Optionally or alternatively, the blade is wider near the entrance to the gap, optionally to prevent tissue escape.

In some exemplary embodiments of the invention, the gap is temporary and is provided, for example, by a gripper which can be opened to receive tissue and then closed. Optionally, a same blade as used for incision is used for cutting tissue in the gripper.

An aspect of some embodiments of the invention relates to a laparoscopic tool in the shape of a rigid hook which is deployable in a direction radial to a tool body. In an exemplary embodiment of the invention, the hook is contained in the tool body and when an overtube is advanced over the hook, the hook is rotated by the overtube engaging a proximal side of the hook. Optionally or alternatively, the hook is extended by pulling a tension element attached thereto.

In an exemplary embodiment of the invention, the hook forms an angle of 90 degrees or less (e.g., 80, 70 or 60 or less) with the tool body, which may prevent tissue from slipping off the hook when the tool body is retracted. In an exemplary embodiment of the invention, the hook is in the form of a bend element with the angle between the two parts matching the angle that the hook forms, when an overtube snugly engages the proximal part of the hook.

An aspect of some embodiments of the invention relates to a clamp that slides along a passage in a tool body and is optionally pulled and/or pushed by the tool body to cause it to change shape. In an exemplary embodiment of the invention, the rigid tube does not apply forces on the clamp. In an exemplary embodiment of the invention, the clamp can be detracted from the tool body and remain in the body.

An aspect of some embodiments of the invention relates to a lasso which is sized to fit between a rigid tube and a replaceable tool. Optionally, the lasso is inserted by grabbing the lasso with a grasping tool and inserting the tool and the lasso into the rigid tube. Optionally, the tool body includes a groove for the lasso.

In an exemplary embodiment of the invention, a lasso is tied by pulling two wires thereof, one that extends along said tool and one that is grasped by said tool head

Similar steps and/or components and/or aspects may be labeled with the same numbering in multiple Figs. Descriptions of options, components, steps and/or aspects with regard to one Fig. apply also to similarly labeled options, components, steps and/or aspects of other Figs.

### Exemplary Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Laparoscopic Access Systems

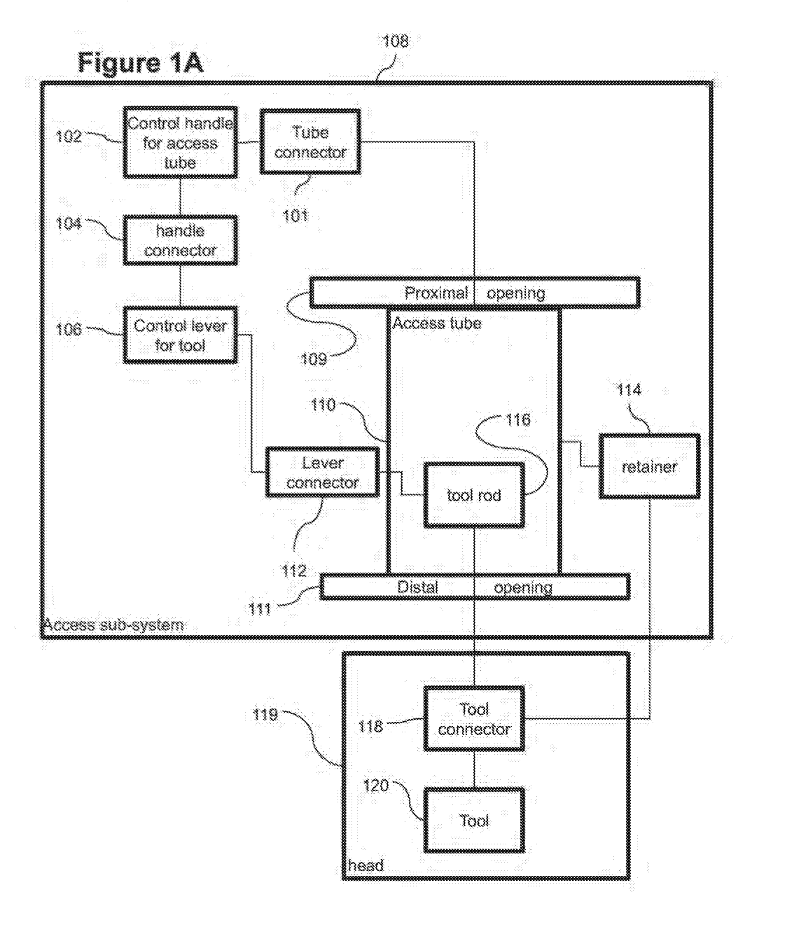

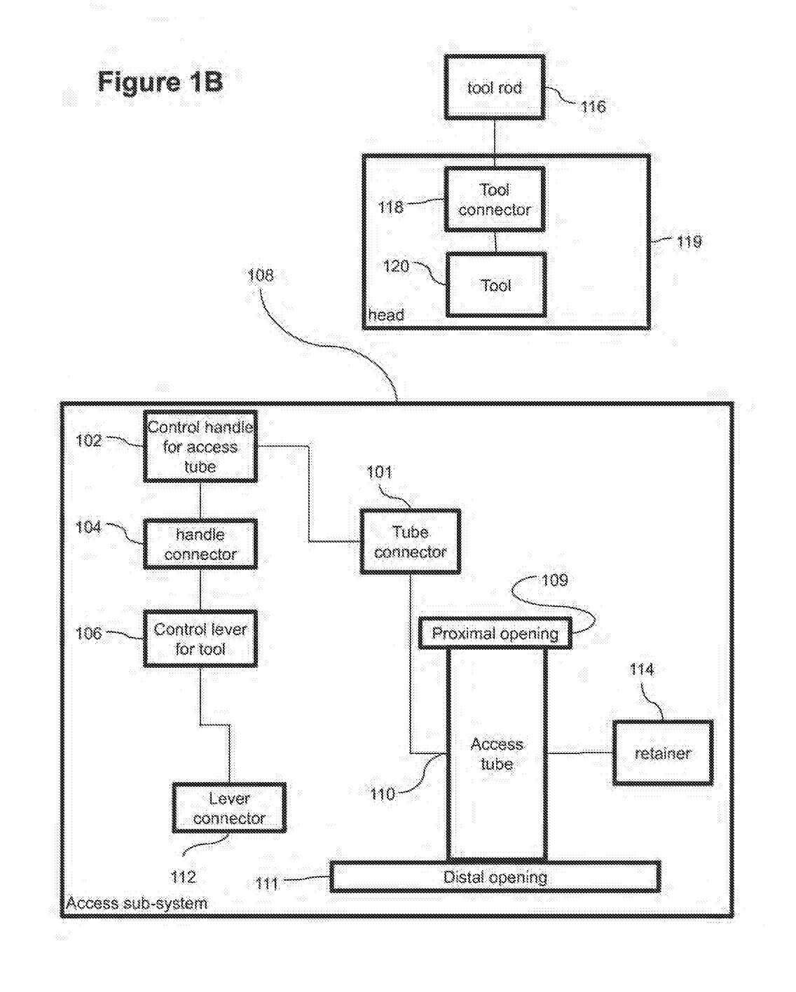

FIGS. 1A-B are a block diagrams of an access sub-system 108 and a tool head 119 in ready state and in a retracted state respectively (for example with head 119 may be retracted tor replacement) in accordance with an embodiment of the current invention. For example, in the ready state an elongated manipulation element, for example a tool shaft 116 is inserted into an access tube 110. Tool shall 116 is optionally attached to head 119 via a tool connector lift. Tool connector 118 may be connected and/or actuate a tool 120. Optionally connector 118 and/or tool 120 protrude from a distal opening 111 of access tube 110. A retainer 114 optionally reversibly locks tool connector 118 to access tube 110. Optionally, movement of the tool shaft 116 with respect to access tube 110 actuates tool connector 118 and/or tool 120. Optionally tool 120 is changed by unlocking retainer 114 and/or disconnected lever 112 and/or retracting tool 120 and/or tool connector 118 through distal opening 111 into access tube 110. Alternatively or additionally, tool 120 and/or tool connector 118 and/or control shaft 116 may be retracted back through a proximal opening 109. Outside proximal opening 109, shaft 116 and/or connector 118 and/or tool 120 may optionally be replaced for example with a different tool.

FIG. 1A illustrates a laparoscopic access system in a ready state in accordance with some embodiments of the present invention. In some embodiments a manipulator, for example a control lever 106 may be connected to tool shaft 116. Lever 106 is optionally moveably connected to access tube 110. The position of and/or movement of shaft 116 with respect to tube 110 may be controlled by positioning and/or moving lever 106.

In some embodiments, the connection between lever 106 and one or both of access tube 110 and/or shaft 116 is reversible. For example when shaft 116 is retracted, it may be disconnected from lever 106 and/or lever 106 may be disconnected from tube 110. In some embodiments, connection and/or disconnection of shaft 116 from tube 110 and/or shaft 116 may be made from a proximal portion of tube 110.

In some embodiments tube 110 is connected on a proximal portion thereof to a handle and/or the handle includes a proximal portion of the tube. The handle optionally includes a handgrip 102. The handle is optionally coupled to tube 110. For example, handle may be rigidly connected to tube 110 for example by means of a tube connector 101. Additionally or alternatively, a tube and handle may be formed together and/or be formed of a single material. Handgrip 102 is optionally used to control the position of tube 110. Optionally, tube 110 is rigid. For example handgrip 102 may be used to control the position of the distal portion of tube 110 from a proximal portion thereof. Optionally, lever 106 is connected via a lever connector 112 to shaft 116. Optionally, lever 106 is connected via a handle connector 104 to a part of the handle, for example handgrip 102. For example handle connector 104 may include a pivot. Optionally, handgrip 102 and/or lever 106 are located on proximal portion of tube 110. For example, in the ready state and/or while tool 120 is protruding from distal opening 111 of tube 110 the positioning and/or actuating of tool 120 may be controlled by controlling handgrip 102 and/or lever 106. Optionally, handgrip 102 and/or lever 106 can both be controlled with one hand. For example, handgrip 102 and/or lever 106 and/or handle connector 104 may have a form like the handle of a pair of scissors.

In some embodiments locking and unlocking retainer 114 may be accomplished from a proximal portion of tube 110. For example, retainer 114 may be located on the proximal portion. Alternatively or additional, retainer 114 may be located on a distal portion of tube 110, but may be locked and/or unlocked from a proximal portion thereof. For example, retainer 114 may include a pin in the distal end of tube 110 and/or tool connector 118 may have a channel that locks onto the pin for example when connector 118 is rotated in the distal portion of tube 110. Optionally, an operator may lock and/or unlock retainer 114 by twisting a proximal portion of shaft 116 thereby twisting connector 118 in the distal portion of tube 110.

In some embodiments, one operator may change a tool while a second operator holds and/or controls handgrip 102 and/or controls the positional and/or movement of the distal portion of tube 110. Optionally, the changing can be done from the proximal portion of tube 110 while the distal portion of the tube remains in an inaccessible location (for example inside the body of a patient). For example, changing a tool may include locking and/or unlocking retainer 114, and/or changing may include connecting and/or disconnecting shaft 116 from tube 110, and/or changing may include retracting and/or inserting head 119 through proximal opening 109, tube 110 and/or distal opening 111 and/or changing may include retracting and/or inserting shaft 116 through proximal opening 109, tube 110 and/or distal opening 111.

FIG. 1B illustrates a laparoscopic access system in a retracted state in accordance with some embodiments of the present invention. In some embodiments, in the retracted state, tool shaft 116 and/or head 119 may be disconnected and/or unlocked from access subsystem 108. Optionally in the retracted state, tool shaft 116 and/or head 119 have been retracted out of tube 110 and/or proximal opening 109.

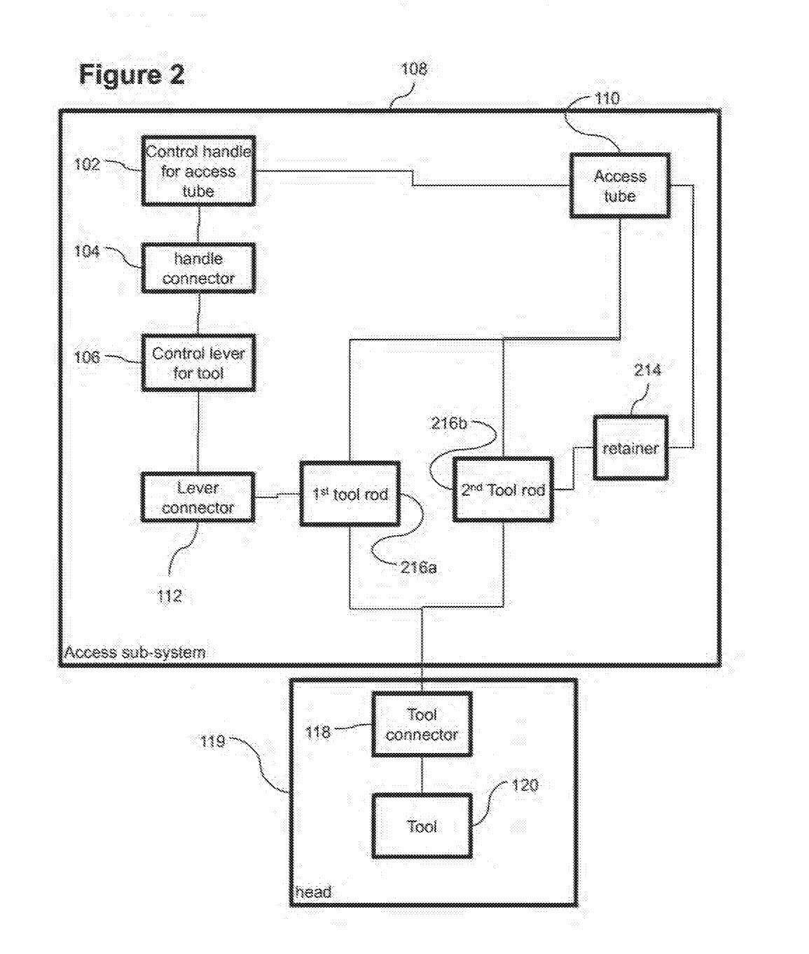

FIG. 2 is a block diagram of a laparoscopic access system with a retainer reversibly locking a second control shaft to an access tube in accordance with an embodiment of the present invention. In the embodiment of FIG. 2, tool connector 118 optionally includes a second shaft 216b. In the embodiment of FIG. 2, tool connector 118 is optionally attached to a first shaft 216a. Actuating of tool 120 may be accomplished for example by moving first shaft 216a with respect to second shaft 216b. For example, in a ready state, shaft 216a may be moveably and/or reversibly connected to control lever 106 while shaft 216b may be reversibly locked to tube 110 by a retainer 214. Optionally controlling the position and/or moving handgrip 102 and/or lever 106 may control the position of and/or actuate tool 120. Optionally, retainer 214 may be located on the proximal portion of tube 110.

In some embodiments, shaft 216a and shaft 216b may be oriented in parallel. For example they may be side by side and/or one inside the other.

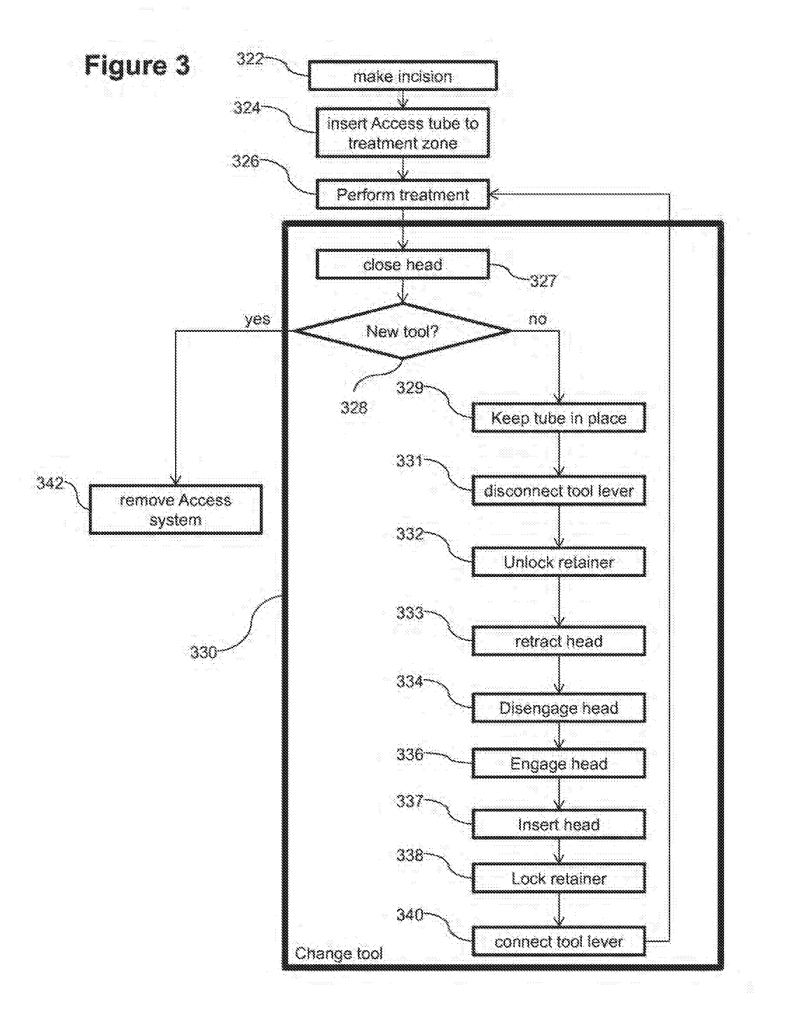

FIG. 3 is a flow chart illustrating a method of deploying multiple tools with a single laparoscopic access system in accordance with an embodiment of the present invention. In some embodiments, the current invention may separate changing 330 of tools from treating a patient (for example treating the patient may include inserting 324 an access tube into the patient and/or performing a treatment 326). A helper may switch tools while a supervising surgeon concentrates on keeping the tube in the correct location and/or performing treatments. Alternatively, or additionally a surgeon may be able insert an access tube once in the patient and use multiple tools in a treatment. Optionally, using multiple tubes with a single access tube may reduce the number and/or size of perforations and/or incisions that are made in a patient. For example an access system of the present invention may fit through an incision of length ranging from 2 to 4 mm and/or from 4 to 6 mm and/or from 6 to 7 mm and/or from 7 to 10 mm. Alternatively or additionally, in some embodiment an access tube may be inserted without a trocar. For example, an access tool may use a smaller incision than a trocar.

In some embodiments, an incision may be made 322 in a patent and/or a distal portion of the access system may be inserted 324 into a cavity (for example the abdomen and/or the pelvis and/or a joint of the patient) and/or a treatment may be performed 326. After performing 326 a treatment and/or when the operator is finished using a tool, the first operator may optionally close 327 the tool head (for example closing a scissors and/or a dissector so that the tool and/or the access system can be safely removed). For example, if there is no desire to insert another tool 328 then the access system may be removed 342 from the patient.

In some embodiments, the first operator may want to insert another tool 328, for example to perform a further procedure. Then the head and/or tool may be removed while the operator retains 329 the access tube in place. For example, an operator may hold a proximal handle of the access tube in order to retain 329 the distal end of the access tube inside the patient and/or within 3 cm of the location of an operation and/or within 1 cm of the location and/or ranging between 3 to 8 cm of the location.

In some embodiments, while the first operator retains 329 the access tube in place a second operator may perform some or all of the actions of changing 330 the tool. For example, a tool control shaft may be disconnected 331 from a control lever and/or a retainer may be unlocked 332. The disconnected tool shaft and/or head may be retracted 333. For example, the tool shaft may be retracted 333 from the distal portion of the access tube in a proximal direction out from the patient and/or out from a proximal opening of the access tube. Optionally, the tool head is connected to the tool shaft. In some embodiments, the tool head may be retracted 333 from outside a distal opening of the access tube through the distal opening into the access tube. In some embodiments, the tool head may be retracted 333 in a proximal direction from the distal portion of the access tube out from the patient and/or out from a proximal opening of the access tube.

In some embodiments, once the tool and/or the tool head and/or the control shaft have been retracted, a tool and/or a tool head may be disengaged 334 from the access system. For example disengaging a tool may include for example removing the tool and/or the tool shaft from the access system and/or disconnecting the tool from the tool shaft. A new tool and/or tool head may optionally be engaged 336 to the access system. The new tool and/or the control shaft may be inserted 337 into the access tube. Insertion 337 may optionally be to a limited (for example by the position of the retainer, the position of the lever and/or the length of the access tube and/or control shaft and/or by a safety system) for example so that the tool is not placed in a position that endangers the patient. Optionally, when the tool, the tool head and/or the control shaft are in position, the tool retainer may be locked 338 and/or the tool shaft may be connected to the tool handle 340.

In some embodiments, after a tool has been changed 330, the first operator and/or another operator, may perform 326 a further treatment. For example the further treatment may include positioning, manipulating and/or actuating the tool with the handgrip of the access tube and/or tool control lever.

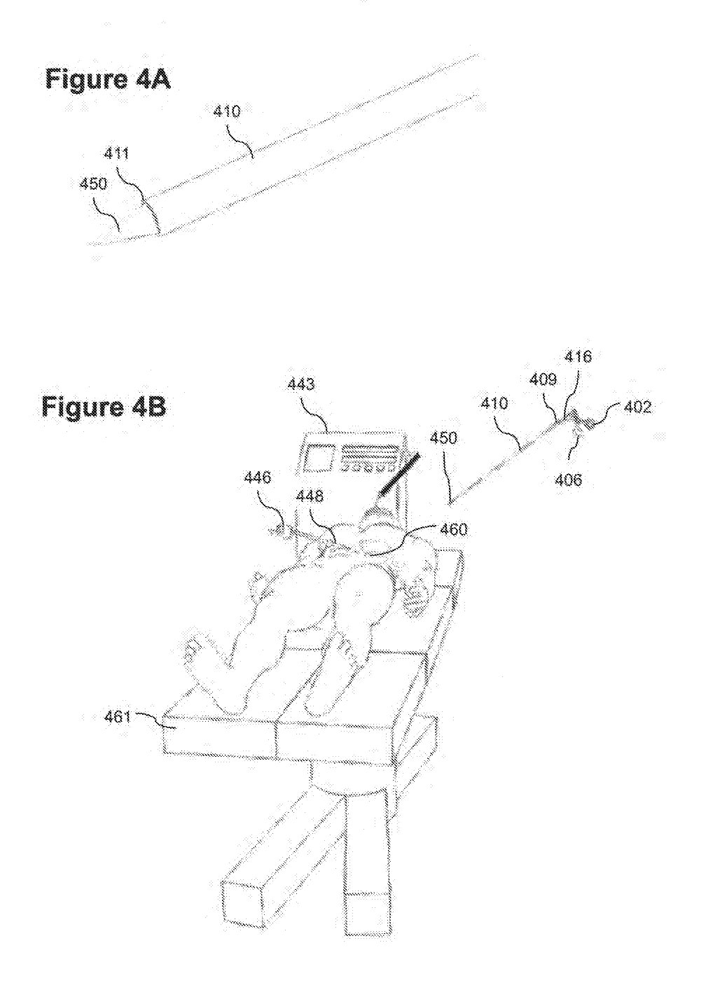

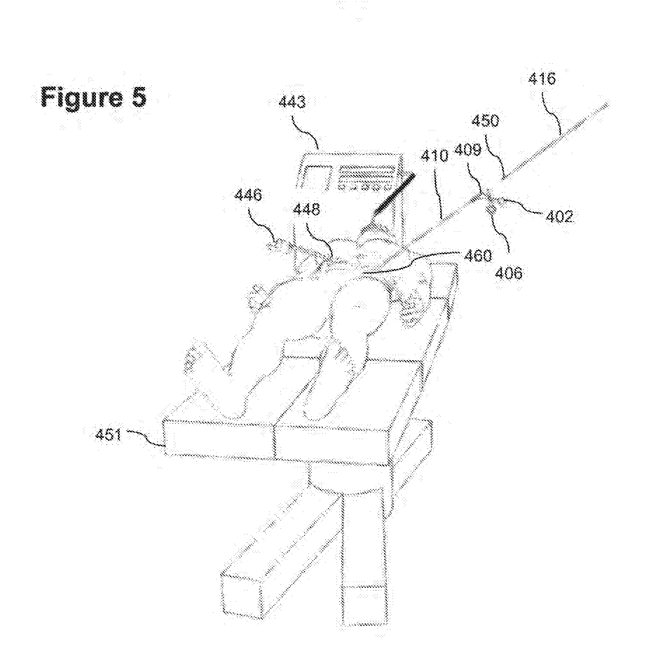

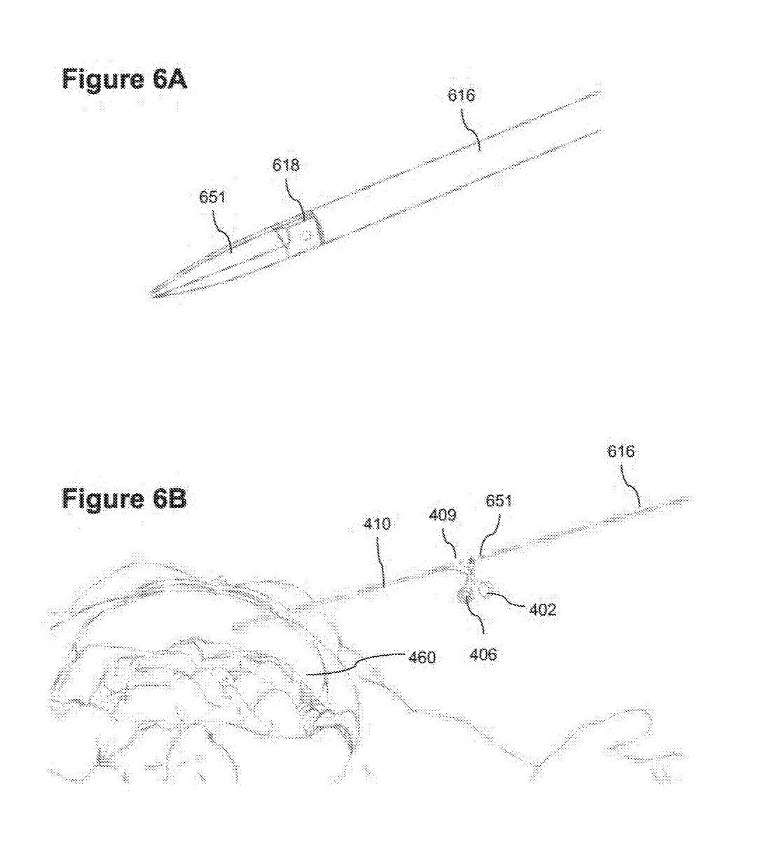

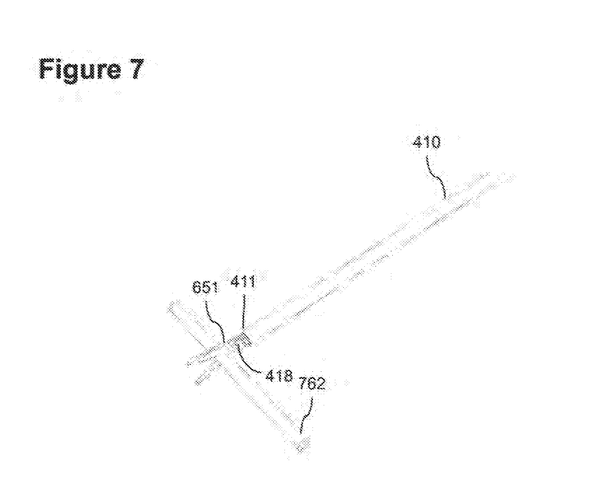

FIGS. 4A-7 illustrate a method of deploying multiple tools with a single laparoscopic access system in accordance with an embodiment of the present invention. The method may include, for example inserting an access tube into a patient, performing an operation with a first tool, changing the first tool to a second tool and/or performing an operation with the second tool.

In some embodiments, a tool may be preloaded into an access tube 410 prior to performing any treatment on a patient. For example, preloading may include changing the tool of the access tool to the needed tool. For example in FIG. 4A, illustrates a distal portion of a laparoscopic access system. For example, a puncture tool 450 has been preloaded into access tube 410. Optionally, puncture tool 450 protrudes from a distal opening 411 of tube 410.

FIG. 4B illustrates an anesthetized patient 460 lying on a surgery platform 451 undergoing a laparoscopic procedure. An anesthesia machine 443 is illustrated. For example a laparoscope 448 including an imaging system 446 is inserted through an umbilicus of the patient. Laparoscope 448 may be used for direct visualization of further procedures. Insertion and/or other procedures may be performed under abdominal insufflations. For example, the insufflations may range from 15 to 25 mm Hg and/or from 25 to 30 mm Hg and/or from 30 to 40 mm Hg.

A laparoscopic access system is illustrated in accordance with the present invention. For example the access system may include an access tube 410. Optionally access tube 410 includes a handgrip 402 and/or a lever 406. Handgrip 402 is optionally connected to lever 406 by a handle connector 404. For example, handle connector 404 may include a pin and/or a rivet and/or a pivot. Puncture tool 450 has optionally been preloaded into tube 410. Puncture tool 450 is optionally attached to a tool shaft, for example rod 416. Puncture tool 450 may optionally be used by a surgeon to insert access system into the insufflated abdomen of the patient. For example, insertion may include making an incision ranging between 2 mm to 4 mm and/or between 4 mm and 5 mm and/or between 5 mm and 6 mm and/or between 6 mm to 7.5 mm and between 7.5 mm and 10 mm and between 10 to 15 mm. After inserting the distal end of access tube 410 into the abdominal cavity an operator may want, for example to change puncture tool 450 for a cutting tool.

FIG. 5 illustrates retracting a punctuate tool 450 and/or tool rod 416 in accordance with an embodiment of the present invention. For example, while the distal end and/or distal opening 411 of access tube 410 remains in the abdominal cavity of patient 460, a surgical assistant unlocks and/or disconnects tool 450 and/or rod 416. For example, while the distal end and/or distal opening 411 of access tube 410 remains in the abdominal cavity of patient 460; a surgical assistant retracts tool 450 and/or rod 416 out of a proximal opening 409 in access tube 410.

FIG. 6A illustrates a distal portion cutting tool 651 (for example a scissors) and a tool rod 616 in accordance with an embodiment of the present invention. Optionally the external diameter of cutting tool 651 and/or tool connector 618 is small enough to fit through tube 410 and/or through distal opening 411 and/or proximal opening 409.

FIG. 6B illustrates insertion of cutting tool 651 and tool shaft, for example rod 616 into access tube 410. For example, while a surgeon grasps handgrip 402 and retains distal opening 411 of axis tube 410 is place in the abdominal cavity, an assistant inserts cutting tool 651 and/or rod 616 through proximal opening 409 and/or in to access tube 410. Cutting tool 651 may optionally be inserted to a distal portion of tube 410. Further protrusion of scissors tool 651 may optionally be performed by the surgeon, for example using lever 406 (for example after the assistant has connected rod 616 to lever 406). Alternatively or additionally cutting tool 651 may be inserted by assistant until cutting tool 651 protrudes out distal opening 411.

FIG. 7 illustrates a performing a medical procedure with scissor tool 651 in accordance with an embodiment of the present invention. Optionally scissors tool 651 is shown protruding from distal opening 411 of access tube 410. For example, scissor tool 651 may be used to cut an artery 762 and/or a lumen and/or an adhesion inside the abdomen of patient.

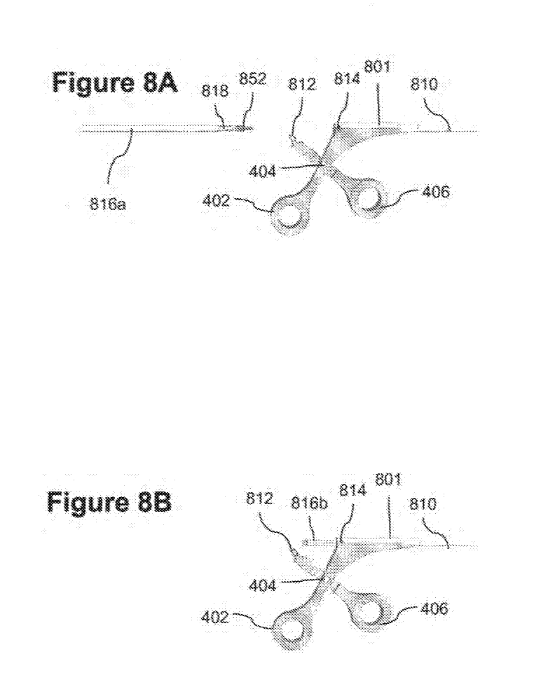

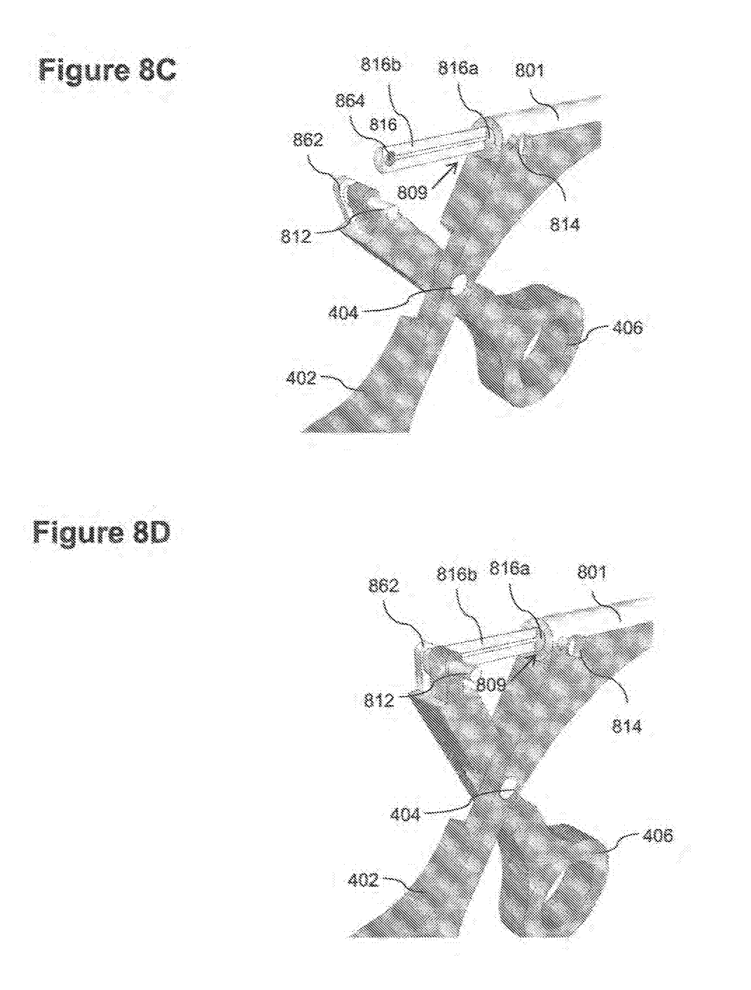

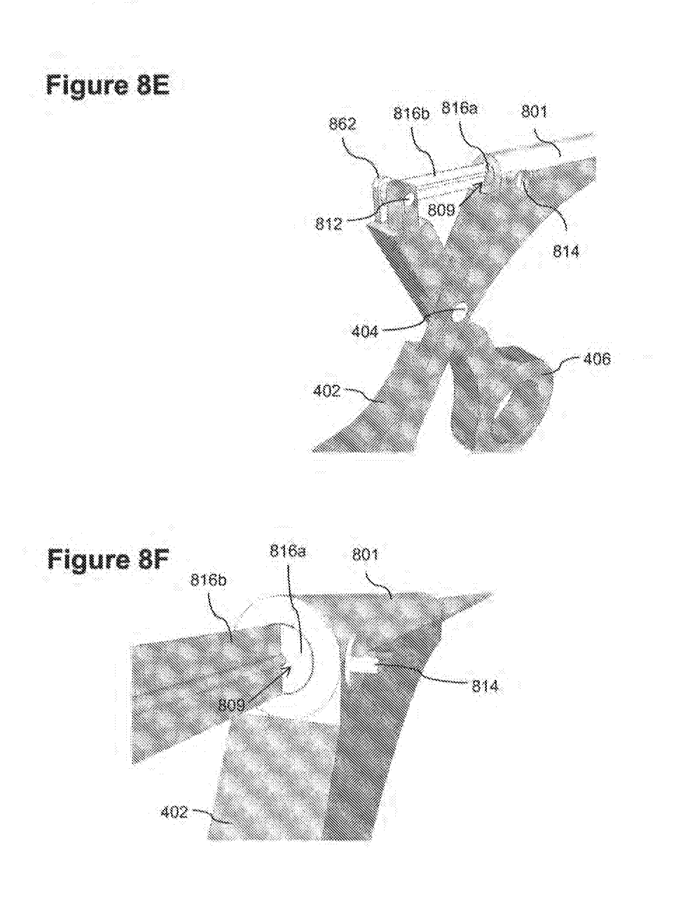

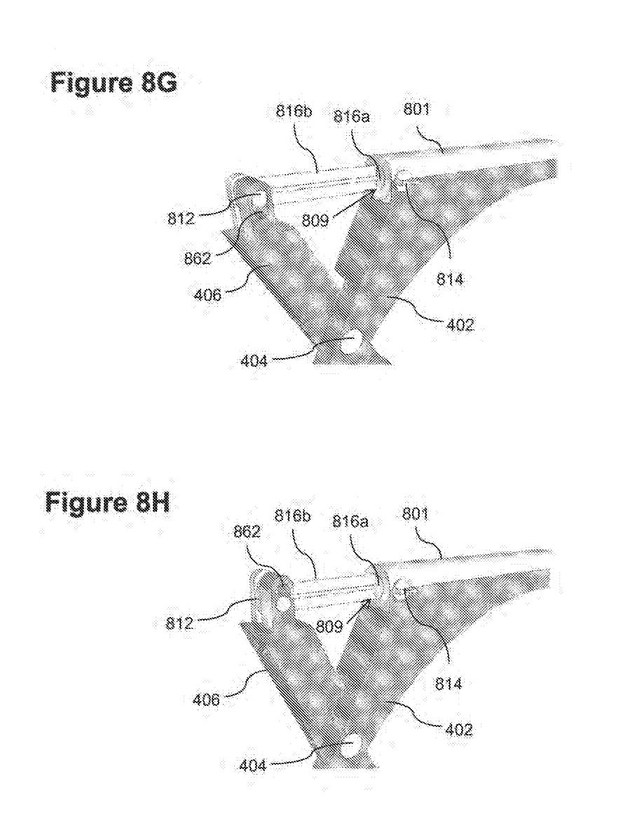

FIGS. 8A-8J illustrate a handle and control lever for a laparoscopic access system with two parallel control shafts in accordance with an embodiment of the present invention. In some embodiments, a tool 852 may be actuated by relative movement between two shafts (for example parallel rods 816a and 816b for example as illustrated in FIG. 8E and 8F. One rod 816a is optionally reversible locked by a retainer. For example, when rod 816a is locked by retainer 814, rod 816a is substantially prevented from moving longitudinally with respect to an access tube 810. Optionally a retainer may be located on a proximal portion of tube 810 and/or the access system. A second rod 816b optionally moves with respect to tube 810 and/or rod 816a. For example rod 816b may be connected to tool control lever 406. An operator may position and/or control movement of rod 816b by positioning and/or controlling movement of lever 406.

FIGS. 8A and 8B illustrate insertion tool 852 and/or a connector 818 and/or of rod 816a and/or rod 816b into access tube 810 in accordance with an embodiment of the present invention (in FIG. 8A. rod 816b is obscured by rod 816a. In FIG. 8B, rod 816a is obscured by the walls of tube connector 801 and/or the walls of tube 810). A tool 852 and/or a connector 818 and/or rod 816a and/or rod 816b may be inserted into access tube through a distal opening. For example the proximal opening may lead to a passage through tube connector 801 which is in communication with the passage of tube 810. Alternately or additionally a tool may be inserted directly into an access tube. Optionally, insertion is limited, for example insertion is until the proximal end of rod 816a is flush with distal opening 809. Optionally, after insertion, a proximal end of rod 816b remains outside of and/or proximal to the passage and/or opening 809.

FIGS. 8C to 8H are close up perspective views of a proximal portion of an access system in accordance with an embodiment of the present inventions. For example FIGS. 8C and 8D illustrate connecting rod 816b to lever 406. Optionally rod 816b includes a mounting hole 864 and/or lever 406 includes a mounting hole 862. An optional level connector pin 812 connects between mounting hole 862 and mounting hole 864. For example, mounting hole 864 may be pivotally connected to pin 812.

In some embodiments, retracting, disconnecting, unlocking, inserting, connecting and/or locking of lever 406, rod 816b, retainer 814 and/or rod 816a may be done by a surgical aid without disturbing the grasp of a surgeon on lever 406, and/or handgrip 402. For example, during the changing procedure, the surgeon can continue concentrating on a video monitor of showing the inside of this patient and/or the surgeon can continue concentrating on keeping tube 810 properly positioned in the abdomen of the patient.

FIGS. 8E and 8F illustrate locking rod 816a to tube 810 in accordance with an embodiment of the present inventions. In some embodiments after connect rod 816b to lever 406, rod 816a may be locked to tube 810. For example a pin of retainer 814 may be inserted through a hole in tube connector 801 into a hole in rod 816a. Locking rod 816a to tube connector 801 may optionally prevent longitudinal movement of rod 816a with respect to tube 810. Alternatively or additionally retainer 814 may be locked before and/or simultaneously to connecting lever 406 to rod 816b.

In some embodiments retainer 814 may indirectly lock rod 816a to tube 810. For example, retainer 814 locks tube 810 to a tube connector 801 that is connects for example to tube 810 in handgrip 402. Alternatively or additionally a retainer may lock a rod directly to and an access tube.

FIGS. 8G and 8H illustrate use of level 406 to move rod 816b with respect to rod 816a in accordance with an embodiment of the present inventions. For example, FIG. 8G illustrates handgrip 402 and lever 406 pivoted away from each other. In some embodiments mounting hole 862 may be elongated, for example allowing play of pin 812 inside bole 862. For example rod 816b may be advanced distally and/or returned proximally without bending.

In some embodiments, an operator may operate and/or operate handgrip 402 and/or lever 406 with one hand. For example, the operator may hold handgrip 402 with his thumb and/or lever 406 with his finger and/or vice versa. Alternatively or additionally handgrip 402 may be controlled using the palm of the hand while lever 406 may be controlled by the fingers or vice versa. Alternatively handgrip 402 and 406 may both be controlled by the fingers. Optionally the thumb may be free to operate a further tool.

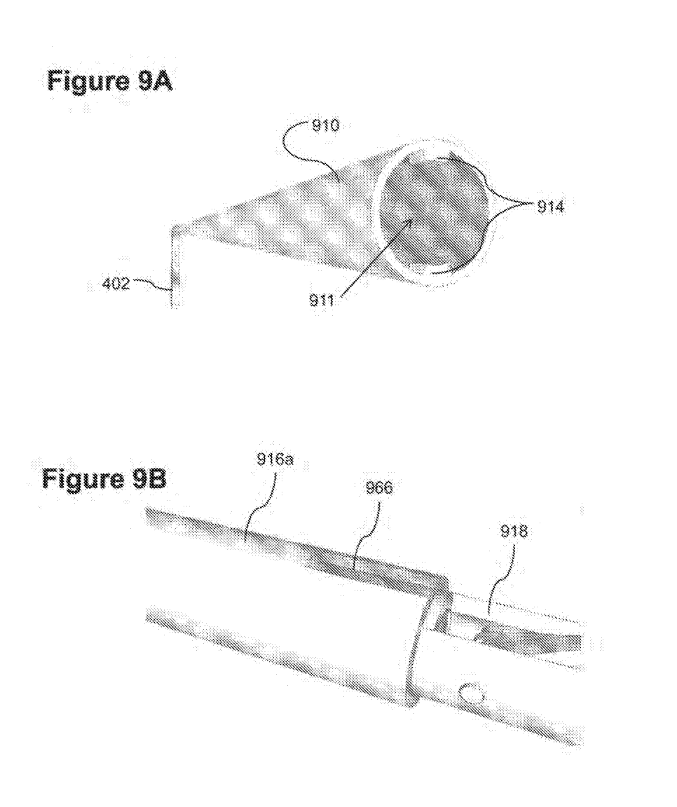

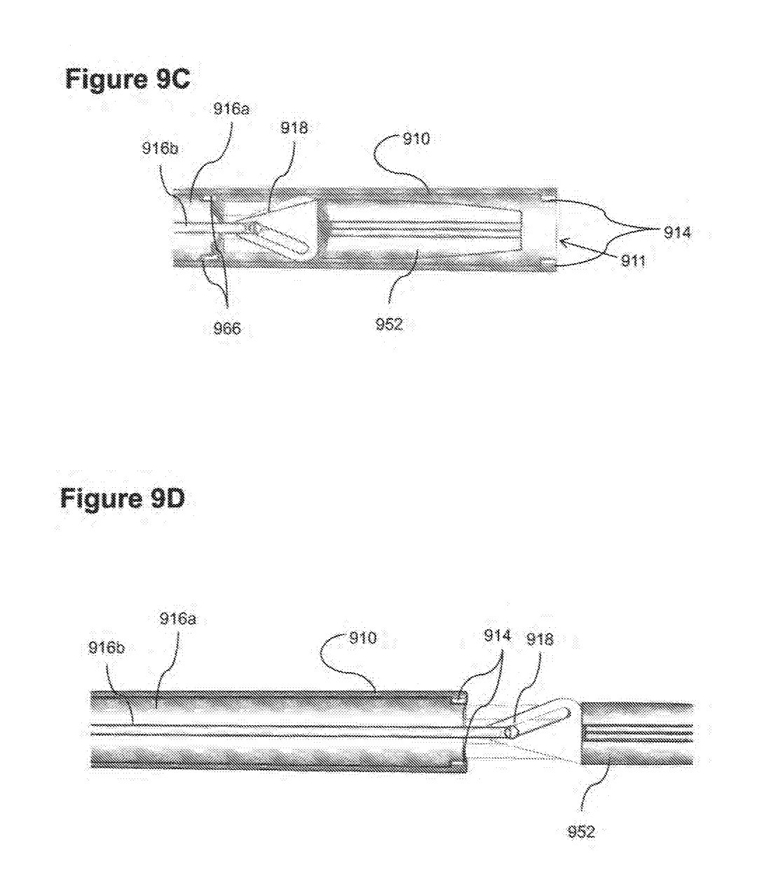

FIGS. 9A-9D illustrate a distal retainer for a tool of a laparoscopic access system in accordance with an embodiment of the present invention. In some embodiments a retainer 914 may be positioned on a distal portion of an access tube 910. For example retainer 914 may include protrusions projecting inward from the inner walls of tube 910. A tool (for example grasper 952) and/or a tool connector (for example connector 918) and/or a tool control shaft (for example tubular rod 916a) optionally includes a track (for example track 966). As the grasper 952 approaches its deployed position (for example at or near a distal opening 911 of tube 910) of track 966 optionally engages protrusions locking the tool and/or a tool connector 918 and/or a tool control rod 916a. Alternatively or additionally, protrusions may be mounted on the tool and/or the tool connector and/or the rod and the track may be located on tube 910. Alternatively or additionally, protrusions may block and/or limit movement of a tool and/or retain the without engaging a track.

FIGS. 9C and 9D illustrate actuation of a tool employing a distal retainer 914 in accordance with an embodiment of the present invention. In some embodiments, tool connector 918 may be connected to an outer shaft, for example rod 916b and/or an inner rod 916a. Movement of inner rod 916b with respect miter rod 916a optionally actuates grasper 952. For example grasper 952 may be biased in a closed configuration and/or grasper 952 may be forced closed while by the walls of tube 910 while grasper 952 is inside tube 910. When track 966 reaches retainer 914 forward advancement of outer rod 916a is arrested. At the same time grasper 952 optionally projects distally out of distal opening 911. Further advancing on inner rod 916b may force open the jaws of grasper 952. Retraction of rod 916b may cause closing of the jaws of grasper 952.

In some embodiments, a track may include a locking mechanism. For example, truck 966 may have an “L” shape and/or a “J” shape. Optionally, when the end of the track reaches the protrusion of retainer 914, an operator (for example a surgical aid) may twist a proximal end of a control rod thereby twisting the track and locking the tool head into place. Twisting the rod in the opposite direction may for example may unlock the tool head and/or retainer. Optionally in some embodiments there may be a single control rod. For example, while the head of the tool is unlocked, advancing and/or retracting the rod may advance or retract the tool inside and/or projecting from the access tube. When the tool head is locked into place, advancing and/or retracting the tool rod may actuate the tool.

In some embodiments a distal retainer and or an access tube may include a safely mechanism. The safety mechanism may limit insertion of the tool into the access tube and/or projection of a tool from a distal opening of an access tube. Limiting distal projection of a tool may prevent injury to a patient by inadvertent deployment. Full distal projection of the tool may be enabled when a control lever and/or retainer are connected and/or locked. For example, subsequent deployment of the tool may be controlled by a surgeon controlling a handle and/or a lever. The safety mechanism optionally prevents over-extension during manipulation of the lever. In some embodiments, the safety mechanism may include narrowing of the access tube and/or the lumen of the access tube, for example on a distal portion thereof.

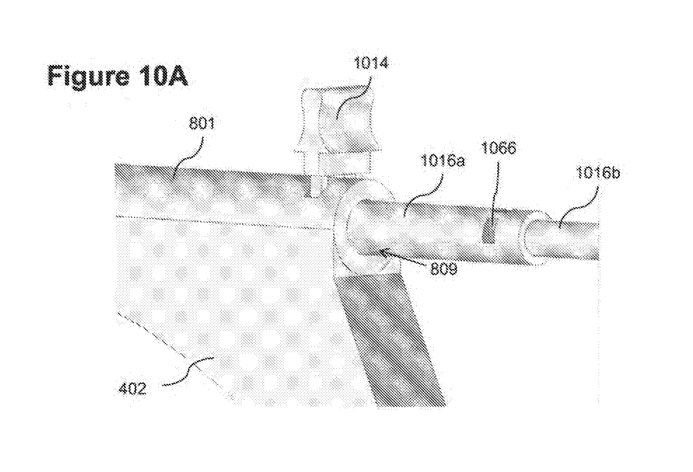

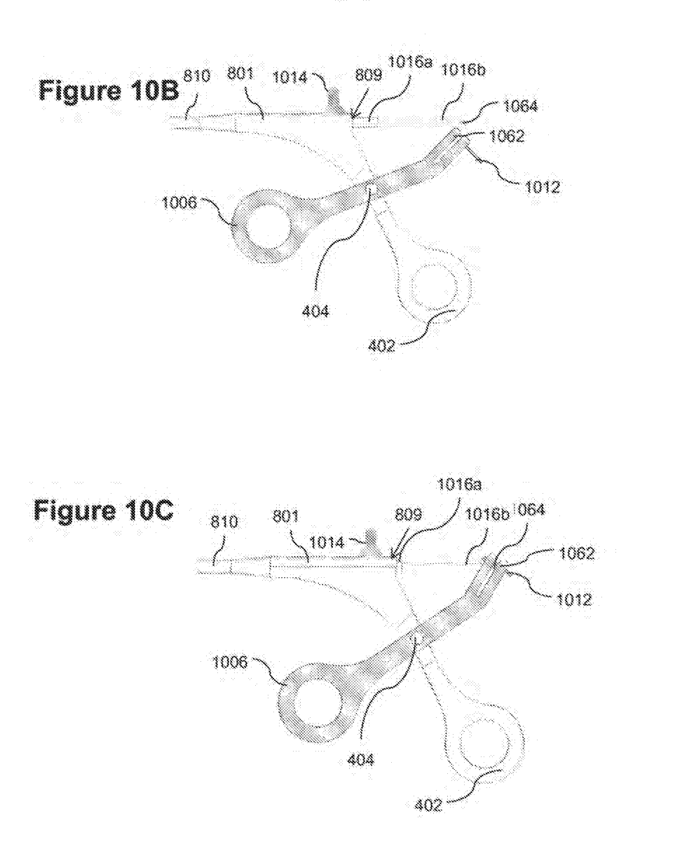

FIGS. 10A-10C illustrate a handle and/or control lever for a laparoscopic access system with inner and/or outer control shafts, for example an inner control rod 1016b and/or an outer tubular control rod 1016a and/or a proximal retainer 1014 in accordance with an embodiment of the present invention. After outer track 1016a has been inserted into the access tube, it may optionally be locked into place by pushing retainer 1014 into a track 1066. Subsequently, movement of inner rod 1016b optionally actuates a tool at the distal end of the rod 1016a and rod 1016b.

FIGS. 10B and 10C illustrate connection of inner rod 1016b to a control lever 1006 in accordance with an embodiment of the present invention. In some embodiments a pair of pin 1012 and pin 1064 connect inner rod 1016b to a track 1062 in lever 1006. Rotating lever 1006 around pivot pin 404 optionally advances and/or returns rod 1016b and/or actuates the tool.

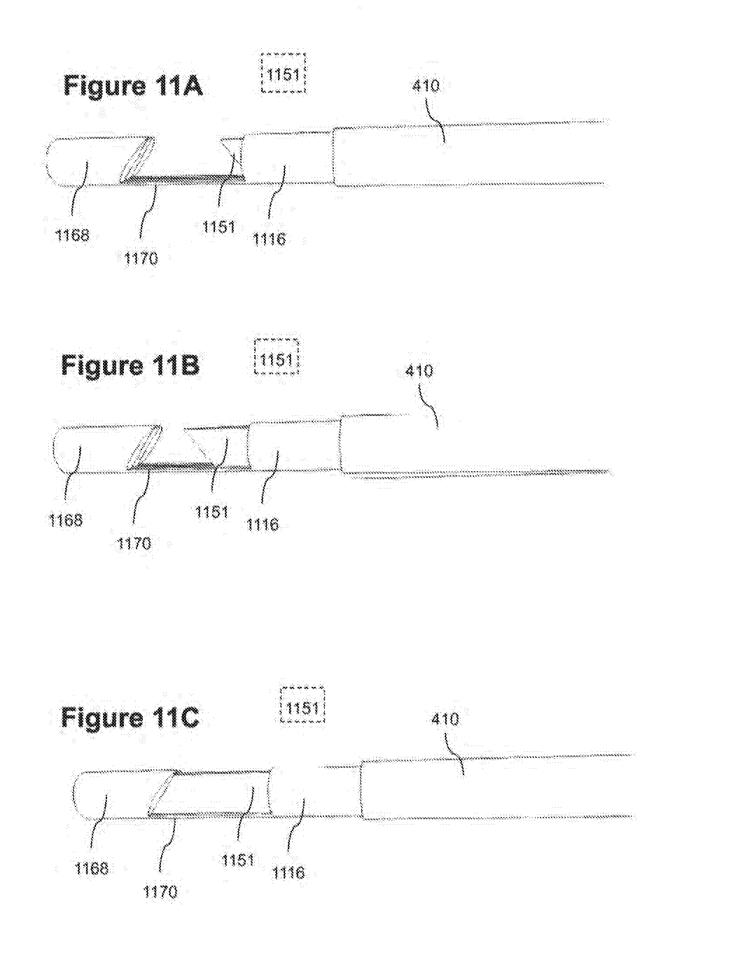

FIGS. 11A-11C illustrate u guillotine cutter laparoscopic tool 1151 in accordance with an embodiment of the present invention. The blade 1151 of the culler may be covered by an outer tool rod 1116. For example, tool 1151 may be deployed and/or moved within a body cavity with reduced danger of unintentionally cutting in inner organ of the patient. When a target is identified, tool 1151 is optionally placed around tool 1151 is moved to position the target in the space defined between the distal cod of rod 1116 and/or the distal end piece 1168 and/or a lateral shield 1170. Once the target is in place, blade 1151 may be advanced to slice the target, for example as illustrated in FIGS. 11B and 11C. Optionally the guillotine cutter may be smaller than the inner diameter of access tube 410. Optionally guillotine culler may be retracted and/or changed as illustrated for other tools for example as illustrated in previous figures. Alternatively or additionally a guillotine cutter tool may have the same diameter as access tube 410 and/or may be deployed using a conventional laparoscopic access system (for example that does not allow changing of tools).

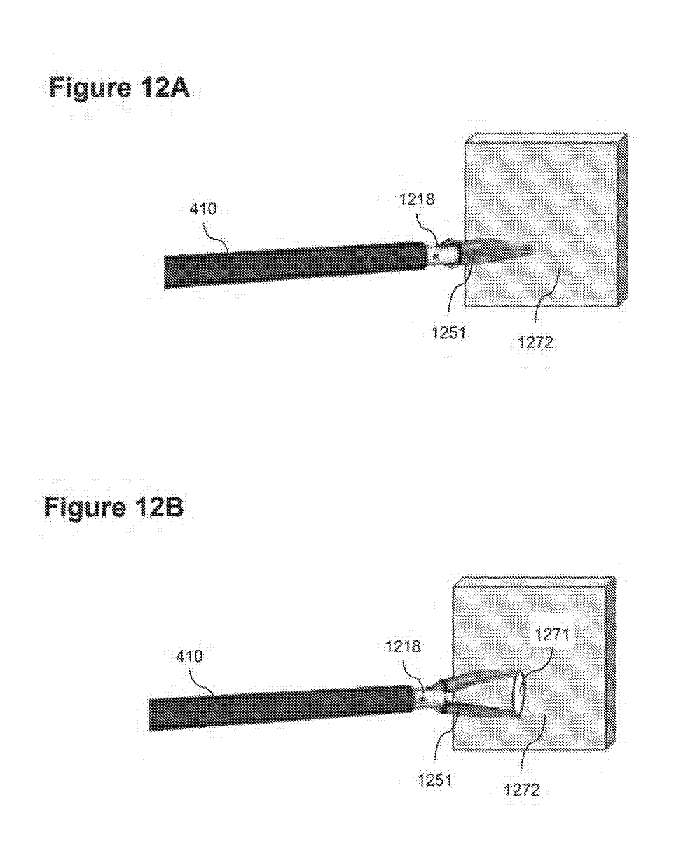

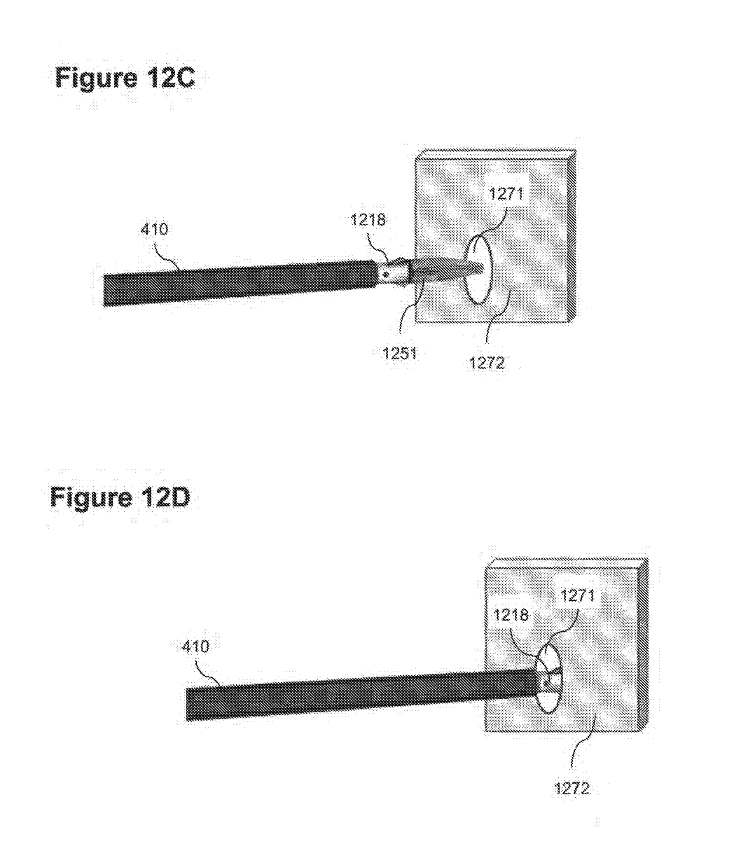

FIGS. 12A-12D illustrate a dissector laparoscopic tool 1251 in accordance with an embodiment of the present invention. In some embodiments of the current invention, tools and/or tool connectors optionally have an outer diameter that is less than the inner diameter of access tube 410. Alternatively or additionally, tool 1251 and/or connector 1218 may have a slightly larger diameter that can be squeezed into tube 410. For example, tool 1251 may be used to puncture a skin 1272 of a patient, for example as shown in FIG. 12A. Optionally tool 1251 may be used to enlarge a hole 1271 skin 1272 of the patient, for example as shown in FIG. 12B. Alternatively or additionally tool 1251 may be inserted through hole 1271 into a cavity of a patient and/or used as an internal dissector and/or grasper, for example as shown in FIG. 12D.

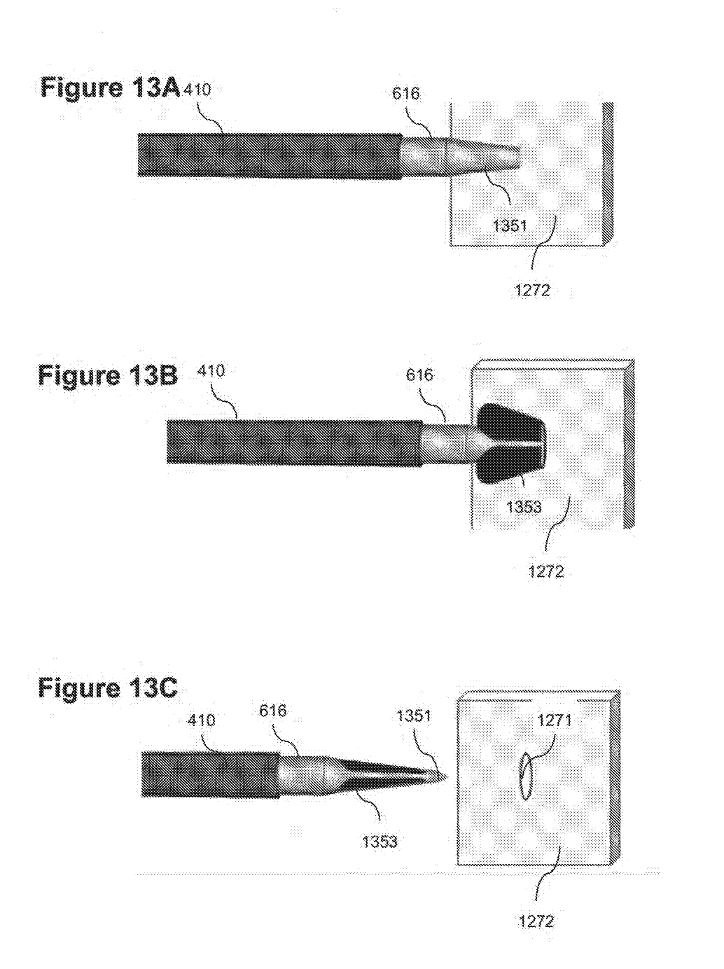

FIGS. 13A-13C illustrate a dissector laparoscopic tool 1351 employing a balloon 1353 to enlarge hole 1271 in accordance with an embodiment of the present invention.

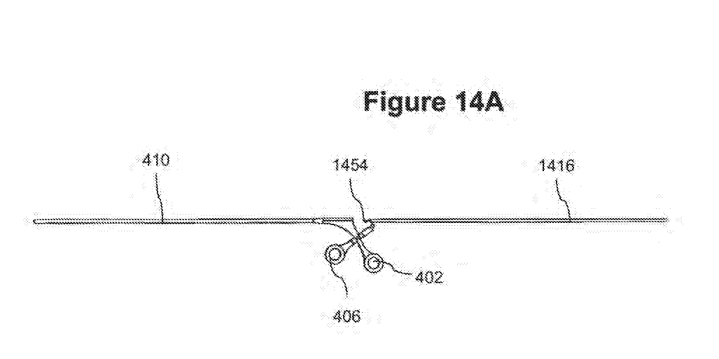

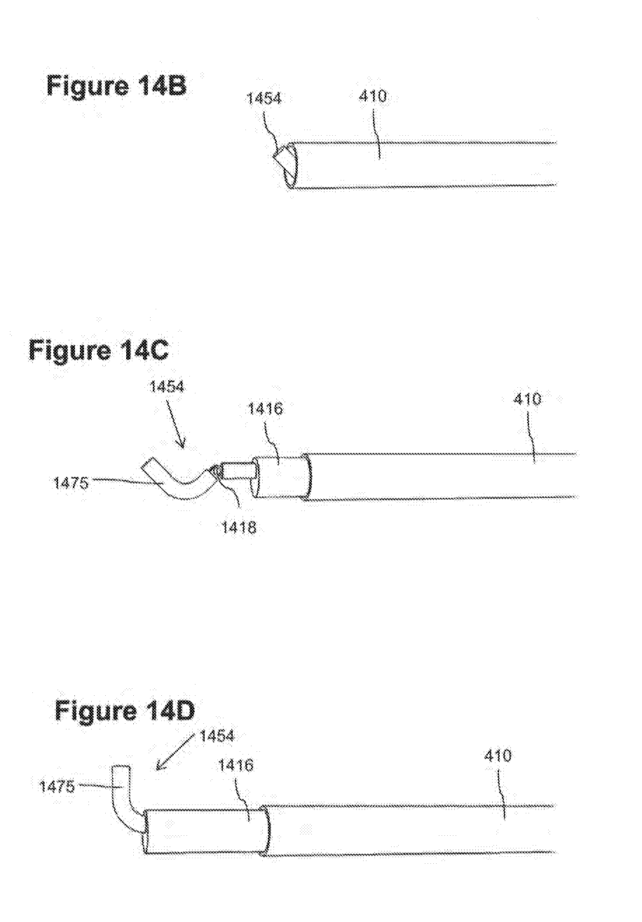

FIGS. 14A-14D illustrates a hook laparoscopic tool 1454 in accordance with an embodiment of the present invention. In some embodiments, hook tool 1454 and optionally be folded (for example using a hinged connector 1418 as illustrated in FIG. 14B). In the folded state, hook tool fits through and/or can be inserted and/or retracted through tube 410 for example for changing a tool. Optionally when an outer rod 1416 is deployed outward (for example using a lever 406) and/or when outer rod 1416 is locked (for example using a retainer) and/or the hook is returned (for example using a lever 406) backwards, the hook is locked in a deployed position for example as illustrated in FIG. 14D. For example, in the deployed position a deployable element, for example a post portion 1475 of the hook stands at an angle ranging between 85 to 95 degrees and/or between 75 to 105 degrees and/or between 55 to 145 degrees to the axis of tube 410 and/or projects beyond the edge of tube 410. For example, post 1475 may project beyond the edge of tube 410 a distance ranging from 1 mm to 5 mm and/or from 5 mm to 10 mm and/or from 10 mm to 20 mm. Hook may be used for example to pull organs into a desired position, for example for cutting and/or suturing etc.

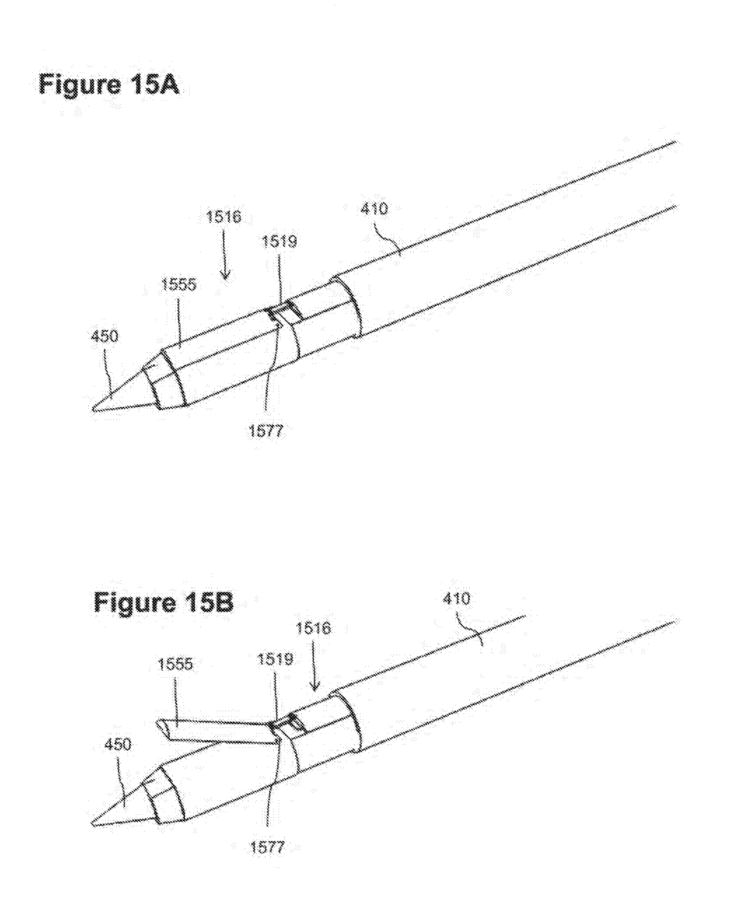

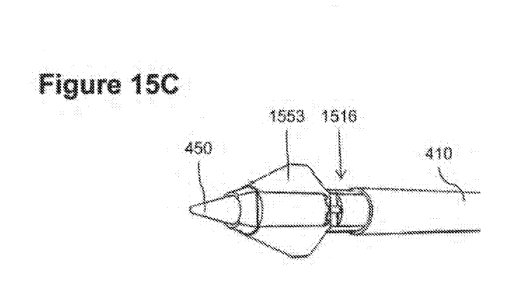

FIGS. 15A-15B illustrate a cauterizer laparoscopic tool in accordance with an embodiment of the present invention. In some embodiments, a cauterizer 1555 is mounted on an outside of a rod 1516. Rod 1516 may optionally be inserted and/or retracted through an access tube 410. For example, cauterize 1555 may be mounted to rod 1516 by a pivot 1577 and/or connected to a wire 1519. In some embodiments, cauterizer 1555 may be biased against the side of rod 1516 for example as illustrated in FIG. 15A. Optionally when an operator pulls wire 1519 proximally cauterizer may pivot away from rod 1516 (for example as illustrated in FIG. 15B). Tissue requiring cauterization may optionally be placed between cauterize 1555 and rod 1516. Then for example heat and/or electricity conducted through wire 1519 can be used to cauterize the tissue. When cauterize 1555 is biased against rod 1516 the tool may optionally fit into an access tube 410 and may be retracted and/or inserted and/or changed.

FIGS. 15C illustrates a balloon dissector laparoscopic tool in accordance with an embodiment of the present invention. For example a balloon 1553 may be mounted on an outside of rod 1516. When balloon 1553 is deflated the tool may optionally fit into an access tube 410 and may be retracted and/or inserted and/or changed.

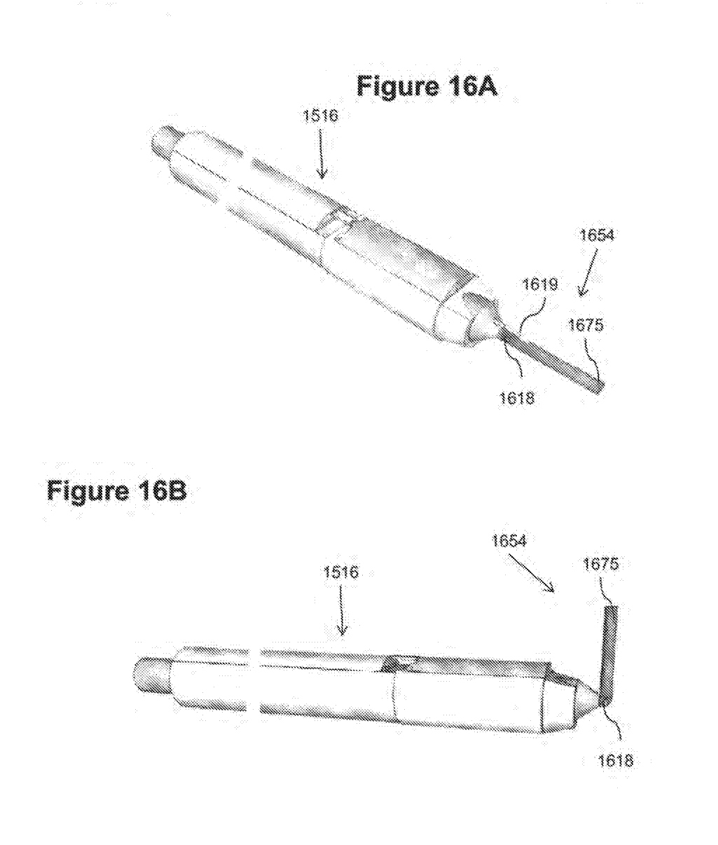

FIGS. 16A-16B illustrate a string actuated hook laparoscopic tool 1654 in accordance with an embodiment of the present invention. A deployable element, for example a post 1675 is optionally connected to a rod 1516 with a hinge 1618. For example, hook tool 1654 may be biased to a straight configuration (for example as illustrated in FIG. 16A) that may optionally fit into an access tube 410 and may be retracted and/or inserted and/or changed. Optionally when an operator pulls a string 1619 (for example using a lever 406 from a proximal portion of the access system) the hook is locked in a deployed position for example as illustrated in FIG. 16B. For example, in the deployed position post 1675 of the hook stands at an angle ranging between 85 to 95 degrees and/or between 75 to 105 degrees and/or between 55 to 145 degrees to the axis of rod 1516 and/or projects beyond the edge of and access tube 410. For example post 1675 may project beyond the edge of tube 410 a distance ranging from 1 mm to 5 mm and/or from 5 mm to 10 mm and/or from 10 mm to 20 mm. Hook may be used for example in pull organs into a desired position, for example for cutting and/or suturing etc.

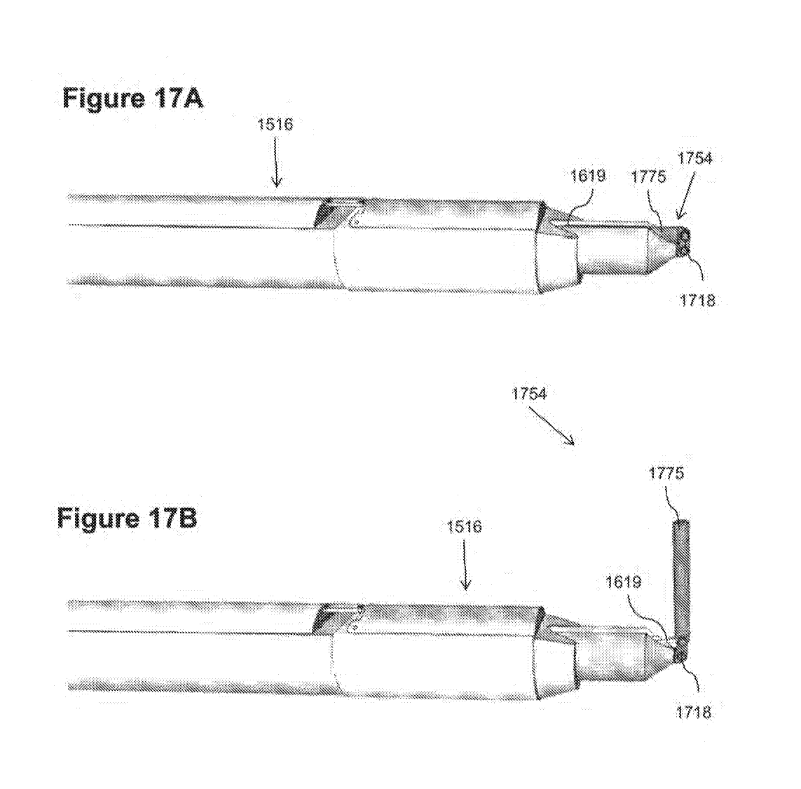

FIGS. 17A-17B illustrate a string actuated hook laparoscopic tool 1754 in accordance with an alternative embodiment of the present invention. A deployable element, for example a post 1775 is optionally connected to a rod 1516 with a hinge 1718. For example, hook tool 1654 may be pulled by a string 1619 to a collapsed configuration (for example as illustrated in FIG. 17A) that may optionally fit into an access tube 410 and may be retracted and/or inserted and/or changed. Optionally when an operator gives slack to string 1619 (for example using a lever 406 from a proximal portion of the access system) the hook is biased to a deployed position for example as illustrated in FIG. 16B. For example, in the deployed position post 1775 of the hook stands at an angle ranging between 85 to 95 degrees and/or between 75 to 105 degrees and/or between 55 to 145 degrees to the axis of rod 1516 and/or projects beyond the edge of and access tube 410. For example post 1775 may project beyond the edge of tube 410 a distance ranging from 1 mm to 5 mm and/or from 5 mm to 10 mm and/or from 10 mm to 20 mm. Hook may be used for example to pull organs into a desired position, for example for culling and/or suturing etc.

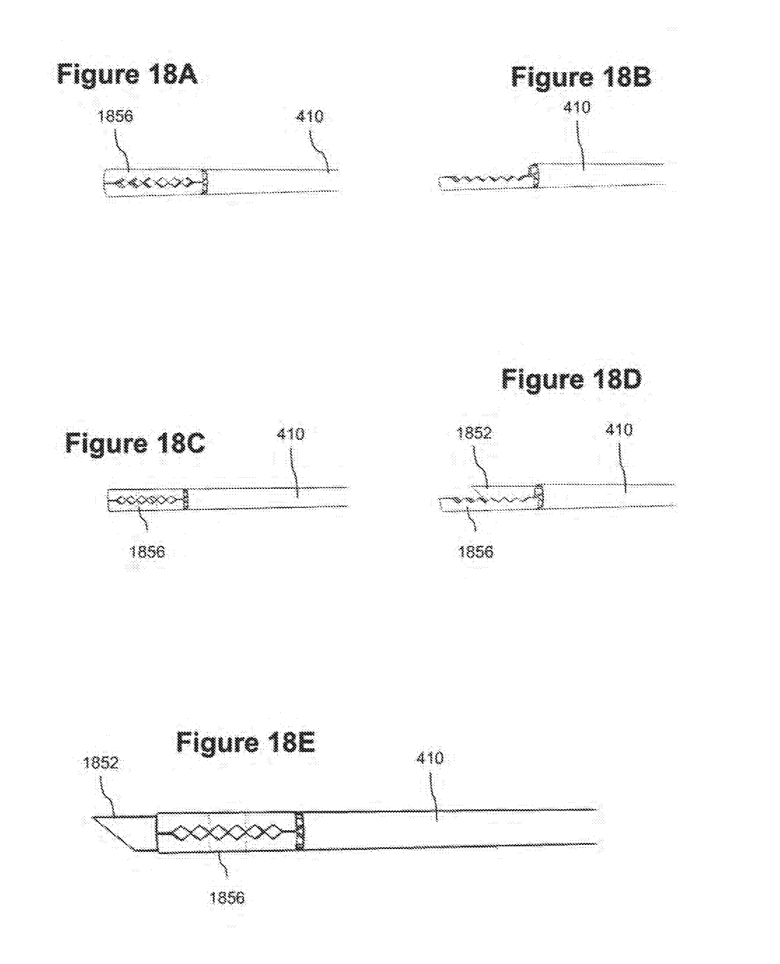

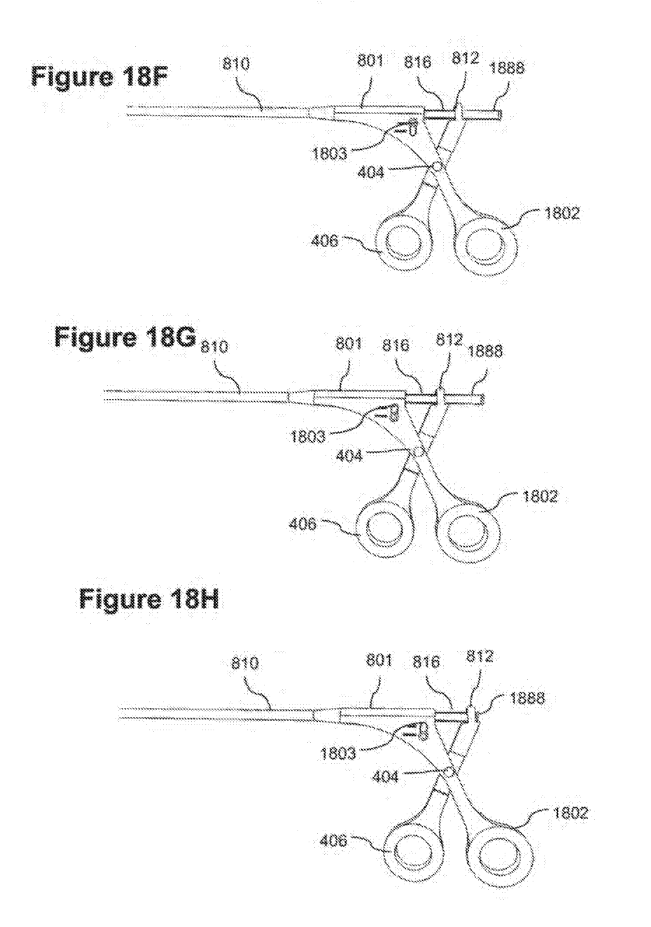

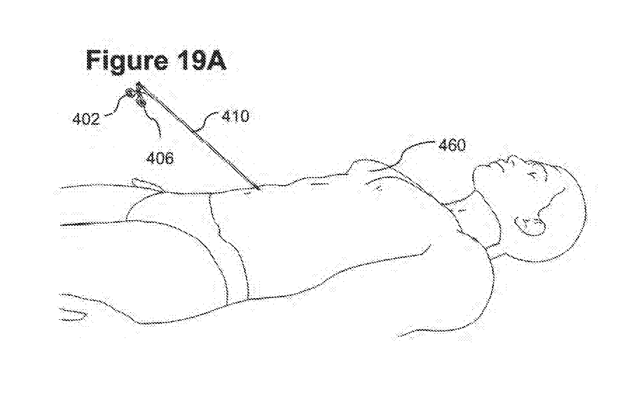

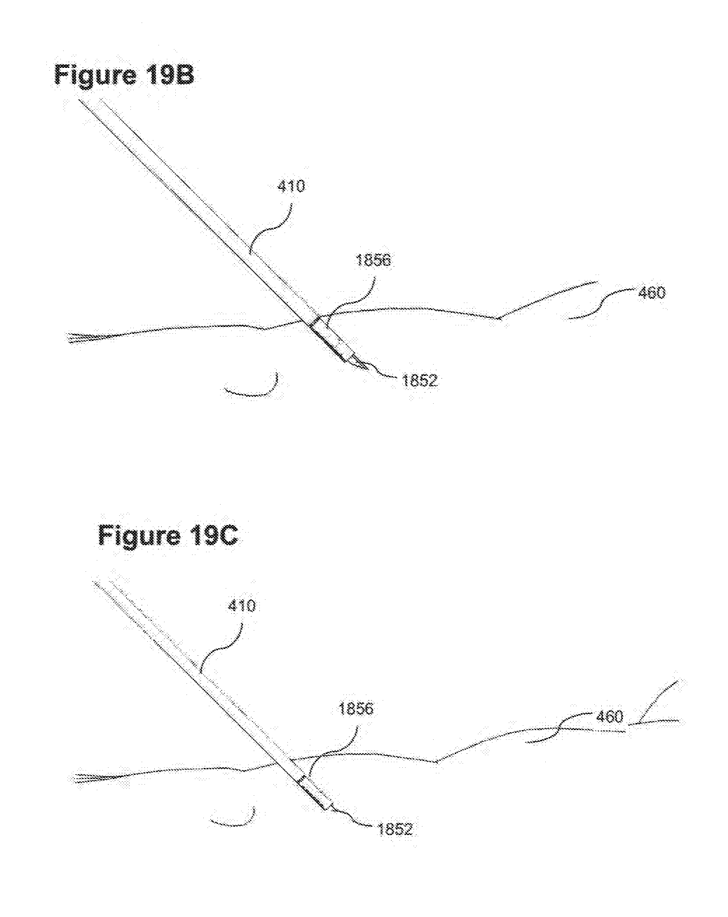

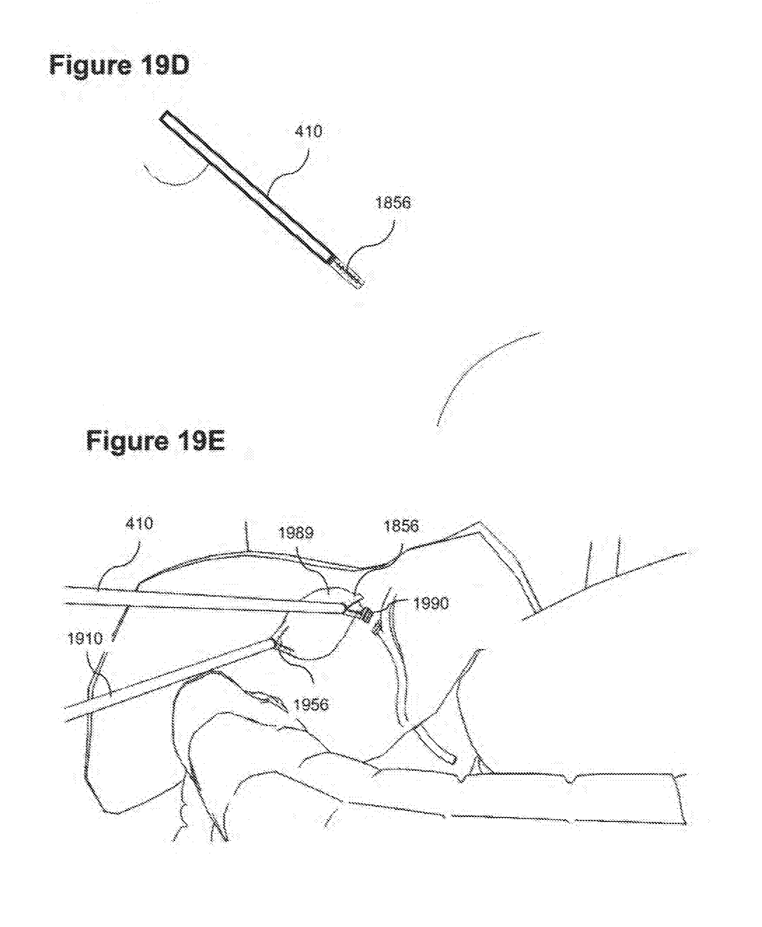

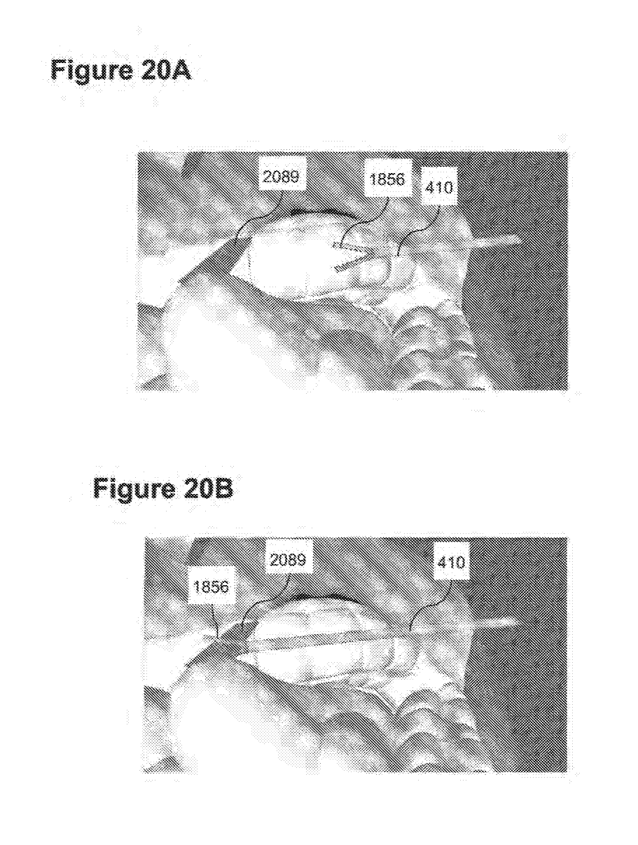

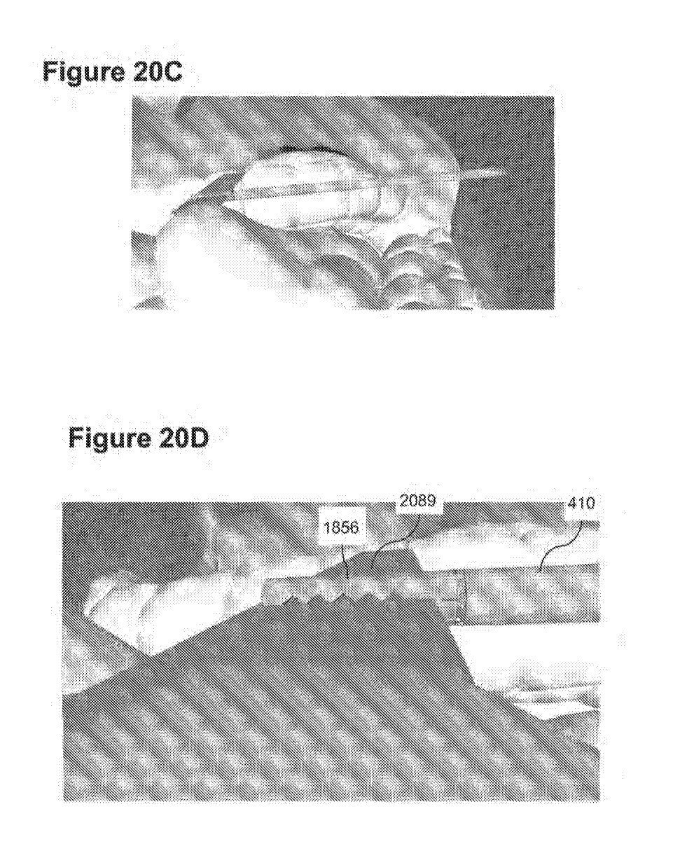

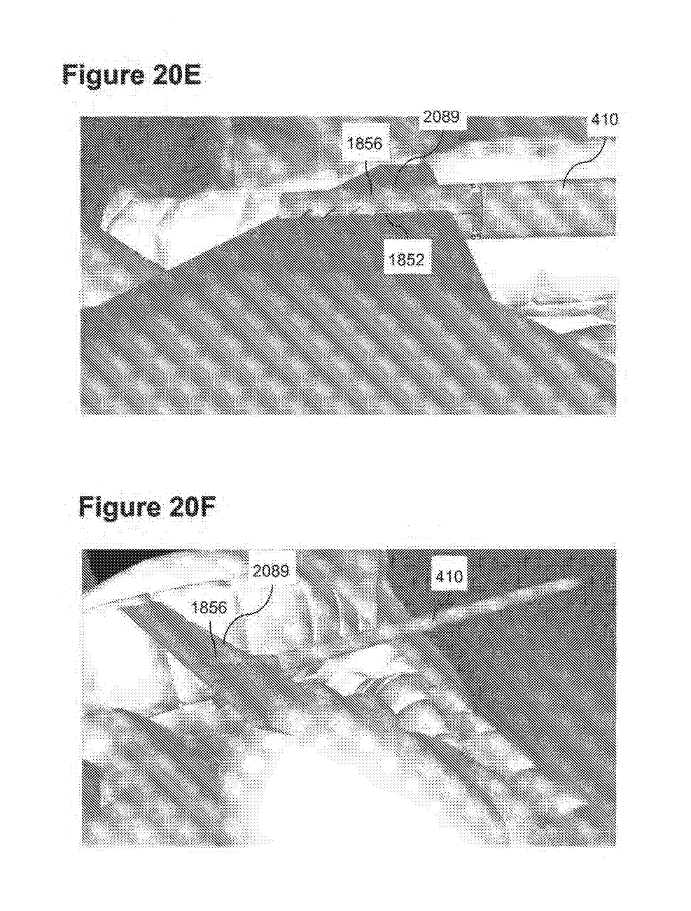

FIGS. 18A-18H illustrate multifunction laparoscopic tool including a cutter and/or another tool in accordance with an embodiment of the present invention. For example the exemplary embodiment of FIGS. 18A-18H includes a grasper 1856 with a cutter 1852 (for example cutting as illustrated in FIG. 20E). Optionally cutter 1852 is also a puncture tool (for example as illustrated in FIG. 19B).

FIG. 18A is a side view of a multifunction tool in with grasper 1856 in a closed state and cutting tool 1852 retracted in accordance with an embodiment of the present invention. In order to illustrate the position of cutting tool 1852, the configuration of FIG. 18A is illustrated in FIG. 18B with the top jaw of grasper 1856 removed.

FIG. 18C is a side view of a multifunction tool in with grasper 1856 in a closed state and cutting tool 1852 in a partially extended state in accordance with an embodiment of the present invention. In order to illustrate the position of cutting tool 1852, the configuration of FIG. 18C is illustrated in FIG. 18D with the top jaw of grasper 1856 removed. As cutting tool 1852 is extended distally, it optionally cuts an object held in the jaws of grasper tool 1856.

FIG. 18E is a side view of a multifunction tool in with grasper 1856 in a closed state and cutting tool 1852 in a fully extended state in accordance with an embodiment of the present invention. In the fully extended state cutting tool 1852 optionally projects distally beyond the distal end of grasper 1856. In the fully extended state cutting tool 1852 is optionally used at a puncture tool. Alternatively or additionally, cutting tool 1852 may be limited in distal movement. For example, the distal end of cutting tool 1852 may be limited to positions proximal of the distal tip of grasper 1856 and or to not extend distally of the distal end of grasper 1856.

FIGS. 18F to 18H illustrate a handle configured for actuating a multifunctional tool in accordance with an embodiment of the current invention. In some embodiments, a secondary extension 1888 connects cutting tool 1852 to a proximal portion of the delivery system. For example, extension 1888 includes a rod inserted inside of a hollow control rod 816. Optionally the handle of the delivery system includes a switch 1803 to impede and/or release distal movement of extension 1888 and/or of cutting tool 1852. When switch 1803 is in the locked position (for example as illustrated in FIG. 18F, cutting tool 1852 is optionally locked in the closed state. Optionally when switch 1803 is in the unlocked position (for example as illustrated in FIG. 18B and/or FIG. 18C) cutting tool 1852 may optionally be moved from the closed position to the partially extended state and/or fully extended state. For example, cutting tool 1852 may be moved distally by pushing extension 1888 distally to a distal position in relation to shall 816. For example an operator may hold a handgrip 1802 with is palm and/or finger and/or push extension 1888 with his thumb. Optionally cutter 1852 is biased to the closed position. Alternatively or additionally, cutter 1852 may be biased to a closed position. Alternatively or additionally, cutter 1852 may returned to a closed position by pulling on extension 1888 proximally.

FIGS. 19A-19E illustrate use of a grasper and/or dissector laparoscopic tool with a cutter to remove a gall bladder in accordance with an embodiment of the present invention.

FIGS. 19A-19C illustrate use of cutting tool 1856 as a puncture tool in accordance with an embodiment of the present invention. For example cutting tool is shown in a fully extended state puncturing and/or creating an incision for inserting access tube 410 into an abdomen of a patient 460.

FIG. 19D illustrates positioning the distal portion and/or end of tube 410 and/or positioning cutting tool 1856 to a treatment region. For example an operator may position the distal portion of access tube 410 and/or grasper 1856 inside the abdomen of a patient 460 by manipulating handgrip 1802 outside the patient 460. For example a gall bladder 1989 is held using a secondary access system 1910 and grasping tool 1956. Grasper 1856 of multifunction tool may be used hold and/or cut off a cystic duet 1990.

FIGS. 20A-20F illustrate use of a grasper and/or dissector laparoscopic tool with a cutter to cut an intestinal adherence 2089 in accordance with an embodiment of the present invention. Optionally, the multifunction tool may be positioned, for example as illustrated in FIGS. 20A and 20B. Optionally consequently, grasper 1856 may be used to grab adherence 2089, for example as illustrated in FIG. 20C and 20D. Optionally consequently, cutter tool 1852 may be used to cut adherence 2089, for example as illustrated in FIG. 20E. Optionally consequently, the multifunction tool may be removed us illustrated for example in FIG. 20F.

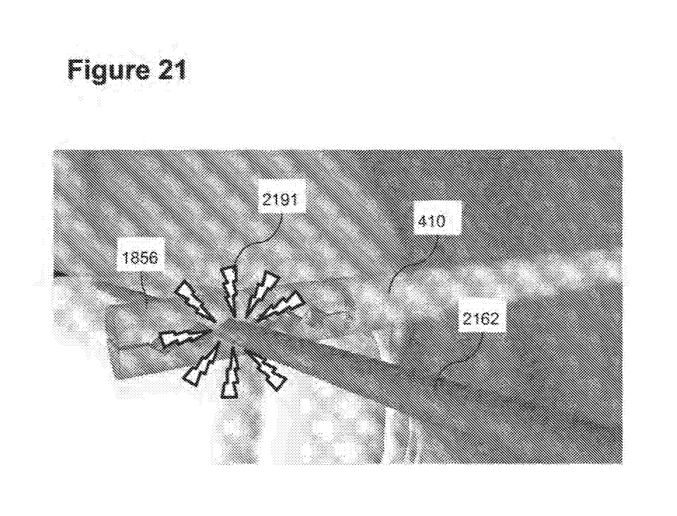

FIG. 21 illustrates use of a multifunction tool with a grasper and/or a cutter and/or cauterization to cut a vessel 2162 (for example a blood vessel) in accordance with an embodiment of the present invention. For example, grasper 1856 may be used to hold vessel 2162. Electricity 2191 and/or a heating element may optionally be connected to grasper 1856. Heat and/or electricity may cauterize vessel 2162. For example, after cauterization, cutting tool 1852 may be used to cut vessel 2162

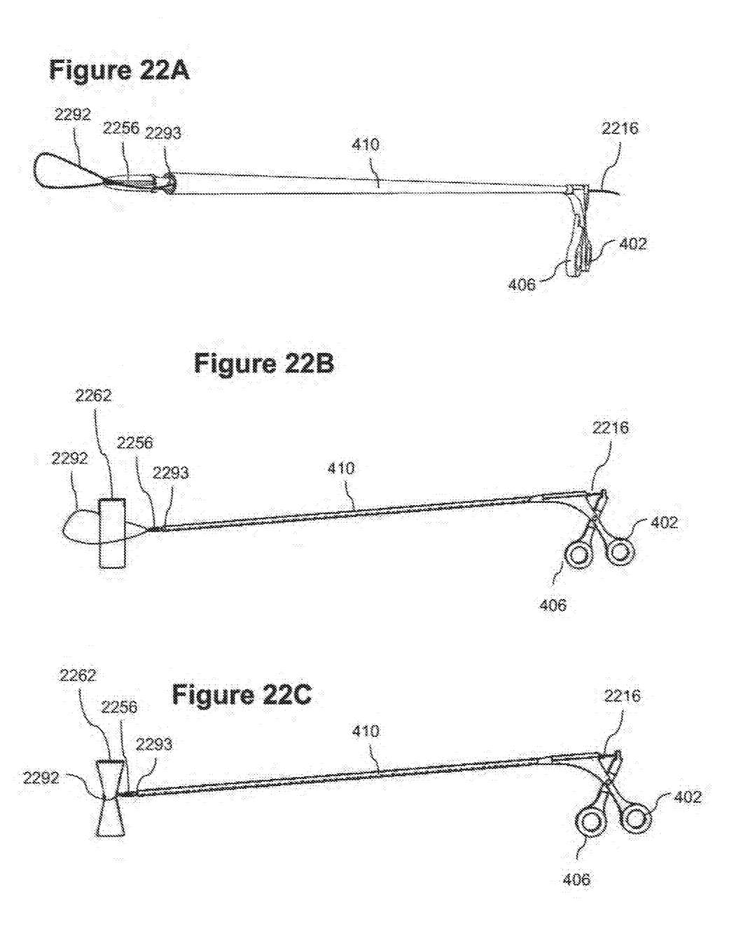

FIGS. 22A-22C illustrate use of a grasper and/or dissector laparoscopic tool to tie off an organ in accordance with an embodiment of the present invention. Optionally the tying procedure can be accomplished with a single access system. For example a loop and/or a lasso 2292 may be tied, for example using the technique of Mishra's knot. Optionally the knot is tied outside the patient. In some embodiments, the knot may have a short end which is placed into the jaws of a grasper 2293. In some embodiments the knot may have a long end 2216 which may be long enough to pass through a channel 2293 in the access system and/or the tool head and/or an extender rod from a distal end and/or portion of the access system to the proximal end and/or opening and/or portion thereof. In some embodiments, the grasping tool 2256 with the loop 2292 may be inserted through an access tube 410 into the patient such that the loop 2292 and/or grasper 2256 protrude out is distal opening into the patient and the long end 2216 extends out a proximal opening of the delivery system (for example as illustrated in FIG. 22A). Optionally, long end 2216 may be connected to a lever 406. Optionally loop 2292 may be placed around an organ 2262 for example is illustrated in FIG. 22B. In some embodiments, loop 2262 may be lightened around organ 2262 by an operator from outside the patient, for example by squeezing lever 406 (for example as illustrated in FIG. 22C).

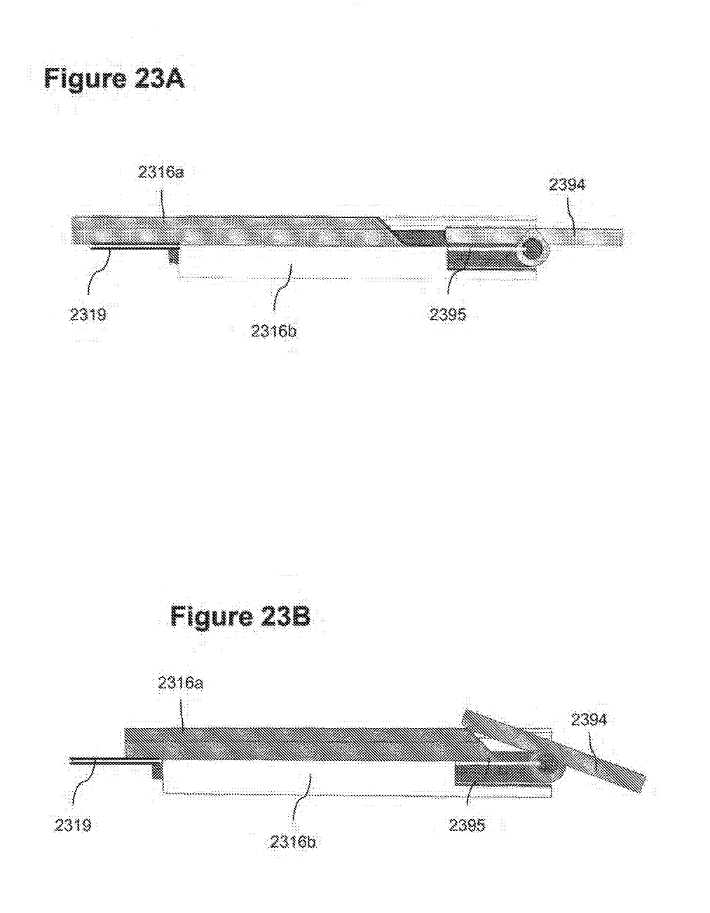

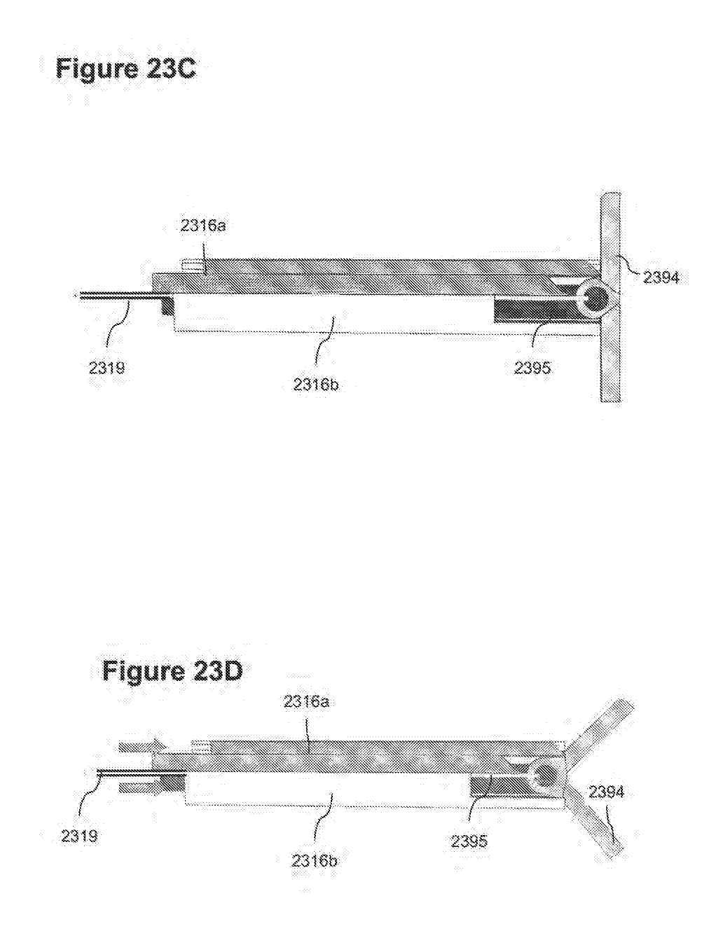

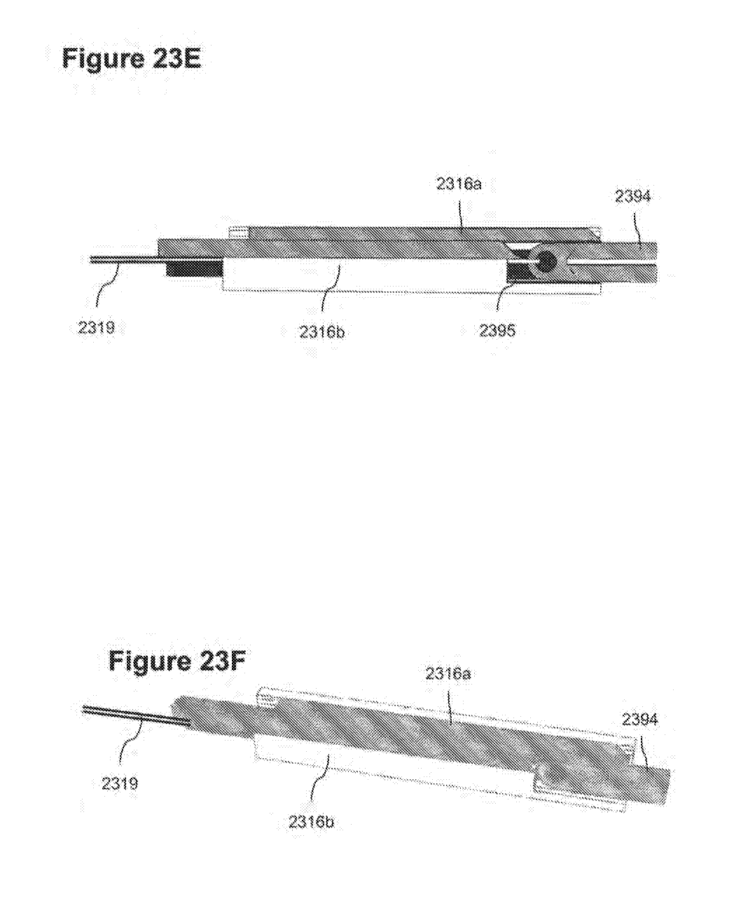

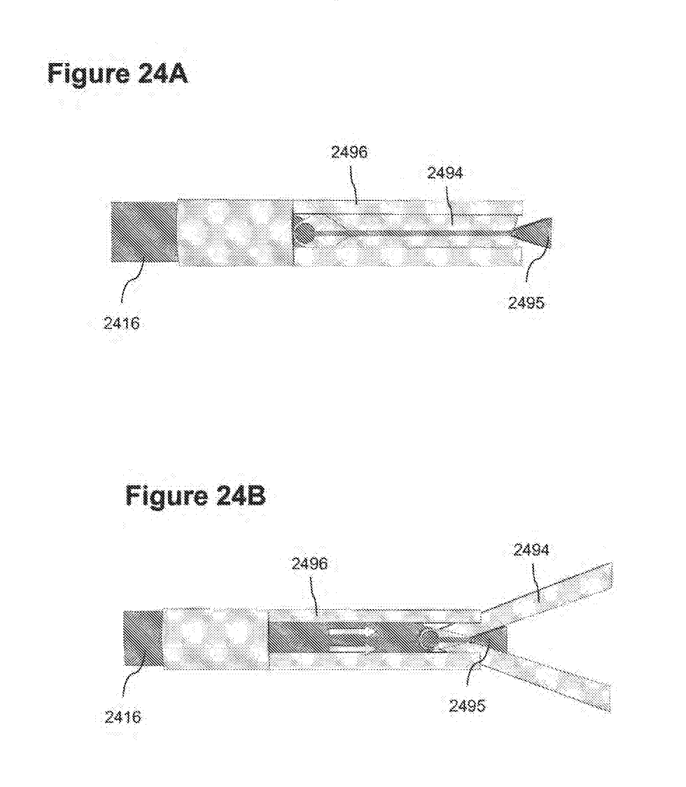

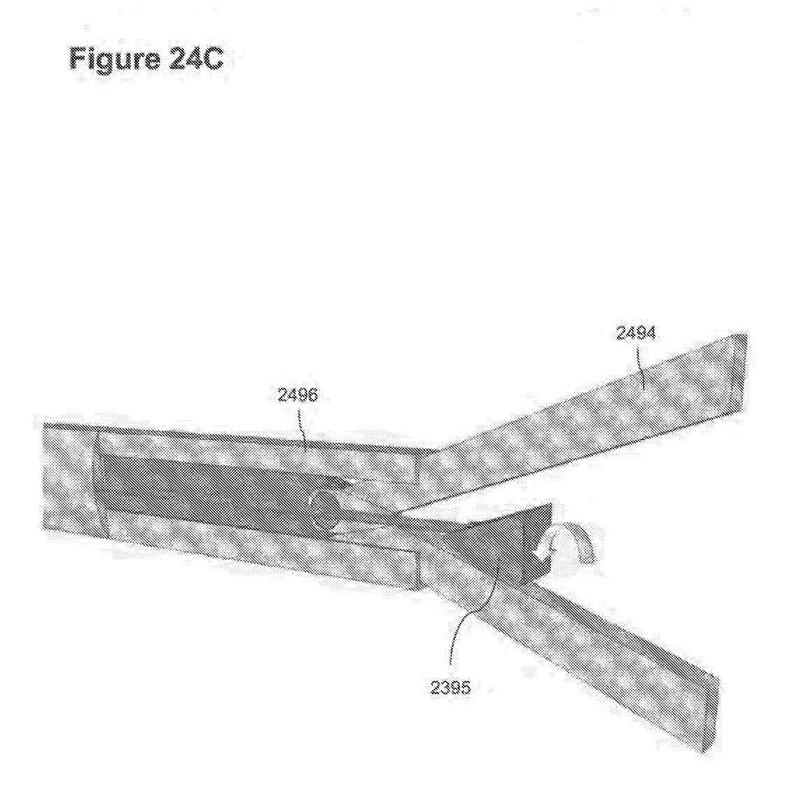

FIGS. 23A-25B illustrate applying a clip in accordance with an embodiment of the present invention. A clip may optionally be left in a patient, for example, after the access system is removed.

FIG. 23A-F illustrate deploying an initially straight clip in accordance with an embodiment of the present invention. In some embodiment, in its initial straight configuration (for example as illustrated in FIG. 23A) a clip 2394 is inserted through an access tube into a patient. Optionally, clip 2394 may be extended out of a distal opening of the access tube and pivoted to a ready position using a control shaft 2316a, for example as illustrated in FIG. 23B and 23C. Clip may be applied to as organ by bending it between two control rods 2316a and/or 2316b for example while it is in the patient (for example as illustrated in FIGS. 23D and 23E). The clip may be released, for example, by pulling a locking bar and/or pin 2395 proximally away from clip 2394 for example with a string 2319 from the proximal end of the access system.

FIGS. 24A to 24C illustrate an alternative clip in accordance with an embodiment of the present invention. For example clip 2494 is biased closed. Optionally clip 2494 is inserted through an access tube and/or a distal opening of the tube into a patient (for example as illustrated in FIG. 24A). Optionally, as clip 2494 is pushed out (for example with a rod 2416) it is pushed over a wedge 2495 and/or wedge 2495 can be pulled for example with a string into clip 2494. Pulling wedge 2495 into clip 2494 optionally opens clip 2494 (for example as illustrated in FIG. 24B). Optionally the access tube is positioned so that clip 2494 surround the organ to be clipped. Wedge 2494 may optionally be take out of dip 2494 for example by rotating and extender and/or wire connected to wedge 2495 (for example as illustrated in FIG. 24C). In some embodiments, clip 2494 then reverts to its biased closed state, clasping the organ.

FIGS. 25A to 25B illustrate an alternative clip in accordance with an embodiment of the present invention. Clip 2594 is optionally inserted into an access tube in an open configuration. Clip 2594 is then pushed out of a distal end of track 2596 for example by a rod 2516. Clip 2594 is optionally biased to an open position (for example as illustrated in FIG. 25A). Clip 2594 is then optionally positioned around an organ and/or closed by pushing track 2496 over the clip (for example by pushing from the proximal portion of the access system with a second rod not shown). Clip 2594 may optionally be released by twisting the proximal end of rod 2516 and/or unscrewing a holder 2595 (for example as illustrated in FIG. 25B). A clip may optionally be left in a patient.

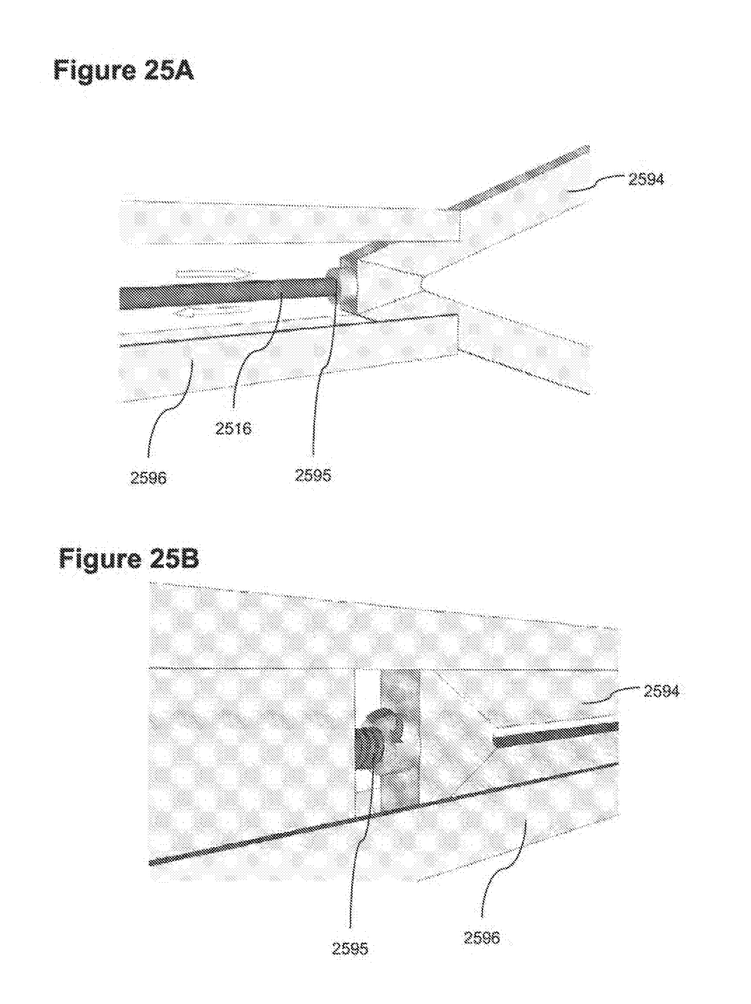

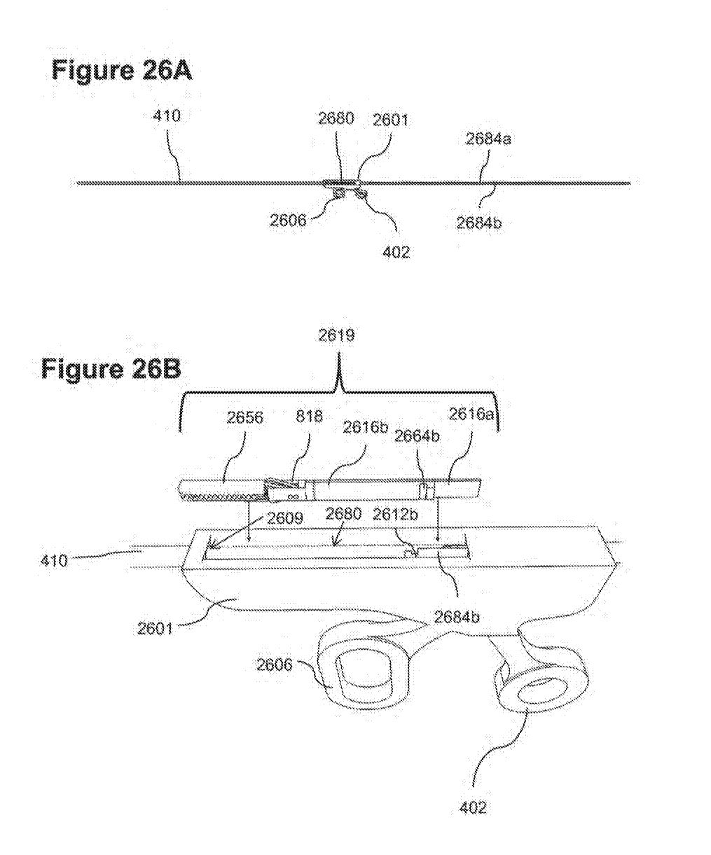

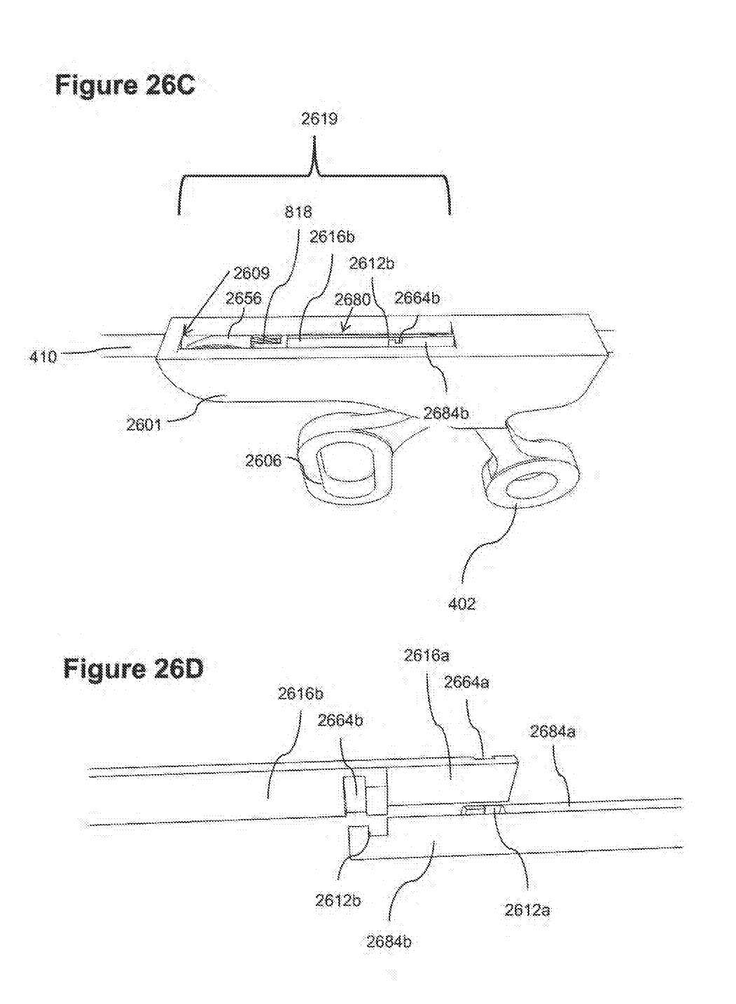

FIGS. 26A-26D illustrate a pistol style handle for a laparoscopic access system in accordance with an embodiment of the present invention. In some embodiments, the tool head may be reversibly attached to one or more elongated tension and/or compression elements (for example shafts and/or rods). The tool may be insertable through a proximal opening of the access tube through the tube and/or out a distal opening of the tube. Optionally a distal portion of shafts may be inserted through the proximal opening or the access tube. Optionally the distal portion of the shafts may connect to the tool head. In some embodiments the tool may be changed by retracting the shafts and/or the tool out the proximal opening of the access tube. Optionally, with the shafts and/or tool outside the access tube, the tool may be disconnected from the shaft and/or a new tool may be coupled to the shaft. The new tool and/or shafts may be inserted into the proximal opening and/or the access tube and/or deployed from the distal opening of the access tube. Optionally a proximal portion of at least one shaft may connect to a manipulator.

FIG. 26A illustrate an access subsystem in accordance with an embodiment of the present invention. In some embodiments a removable head may be loaded into an aperture in the access system handle. For example, a tube extender 2601 portion of the handle may include a loading aperture 2680 and/or one or more elongated extenders, for example rod 2684a and/or rod 2684b. The removable head may be loaded into aperture 2680 and/or pushed distally through access tube 410 and/or actuated by rod 2684a and/or rod 2634b. Loading aperture is optionally proximal to a proximal opening 2609 of access tube 410.

FIGS. 26B and 26C illustrate loading a detachable head 2619 into an access subsystem in accordance with an embodiment of the present invention. In some embodiments, a removable head 2619 may be inserted and/or removed from the access system. The head is optionally reversible attached to extenders that push the head into the access tube and/or retract the head from the access tube and/or actuate a tool.

In some embodiments, a removable head 2619 may include a tool (for example grasper tool 2656) and/or a connector 818 and/or one or more couplings. For example, attachment couplings may include a short rod 2616a and/or a short rod 2616b. Relative movement of short rod 2616a and short rod 2616b optionally actuates tool 2656 (for example grasper tool 2656) and/or a connector 818. Optionally an entire tool head 2619 fits sideways (e.g. perpendicular to the longitudinal axis and/or transaxially) through aperture 2680 in tube handle (for example into tube extender 2601). For example, rod 2684a and/or 2684b may be retracted to a position proximal to aperture 2680. With rod 2684a and/or rod 2684b in the retracted position, head 2619 may be inserted into aperture 2680. As head 2619 is inserted, short rod 2616a may optionally be attached to extended rod 2684a and/or short rod 2616b may optionally be attached rod 2684b. Optionally, while rod 2684a and/or rod 2684b are retracted they may remain moveable attached to the handle for example tube extension 2601.

In some embodiments, after inserting a head (for example head 2619) into aperture 2680 and/or connecting head 2619 to extender rod 2684a and/or rod 2684b, then extender rod 2684a and/or rod 2684b may be used to push head 2619 distally into, access tube 410 and/or out of a distal opening of axis tube 410. For example, a distal portion of tube 410 may be located inside a patient and extender rod 2684a and rod 2684b may be used to push tool 2656 out a distal opening into the patient. In some embodiments an operator may be able to move rod 2684a relative rod 2684b. For example, rod 2684a may be locked to and/or immobilized with respect to tube 410, for example by a retainer. Rod 2684b is optionally attached to lever 2606. Squeezing together lever 2606 and handgrip 402 may cause rod 2684b may be moved distally with respect to 2684a. Moving rod 2684b distally with respect to 2684a may optionally push short rod 2616b distally and/or pull short rod 2616a proximally and/or move short rod 2616b distally respect to short rod 2616a. Optionally relative pushing and pulling are performed simultaneously. Relative movement of short rod 2616b distally with respect 10 short rod 2616a optionally actuates tool 2656.

A lifter may be included in to help remove head 2619 from aperture 2680. For example a flexible strip (for example a string and/or a ribbon) may be placed under head 2619. Pulling up on the ribbon may push head 2619 out of aperture 2680. Alternatively or additionally a mechanical lifter may include a post and/or a cradle and/or a gear and/or a twist knob.

FIG. 26D illustrates a mechanism in connect a tool head to two extenders in accordance with an embodiment of the present invention. For example, short rod 2616a is connected to extender 2684a and/or short rod 2616b is connected to extender 2684b. For example a latch 2664b on short rod 2616b may connect to a latch 2612b on extender rod 2684b and/or a latch 2664a on short rod 2616a may connect to a latch 2612a on extender rod 2684a. Optionally, inside access tube 410 head 2619 and rod/or 2684a and/or rod 2684b are held together by the inner walls of tube 410.

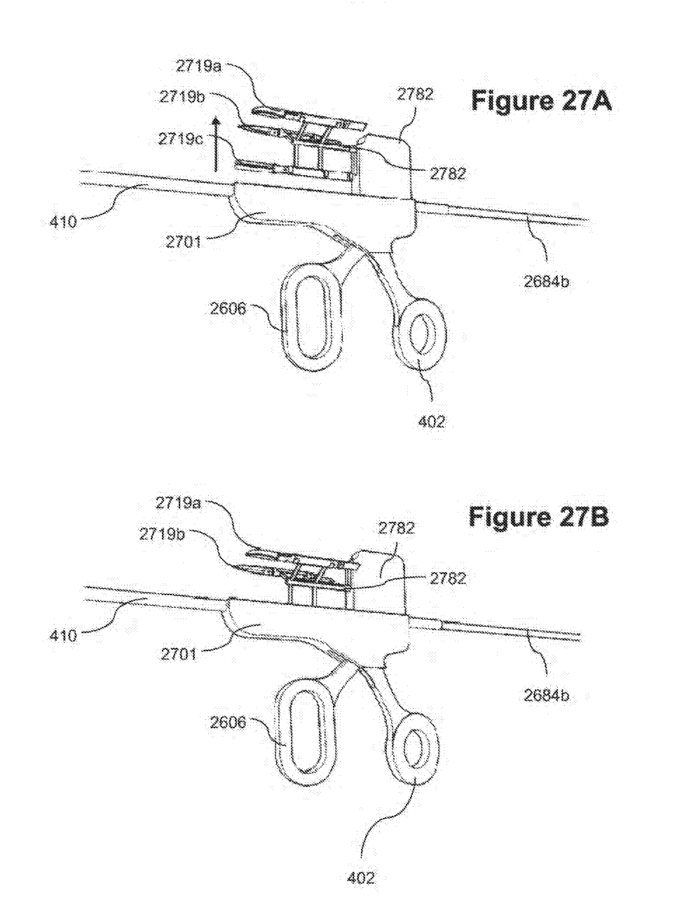

FIGS. 27A-27B illustrate a revolver style handle for a laparoscopic access system in accordance with an embodiment of the present invention. For example, a revolving magazine 2782 is held by a lifter 2782. Magazine 2782 may be lifted to a disengaged position away from a loading aperture in the top of the handle and/or tube extension 2701 (for example as illustrated in FIG. 27A). While magazine 2782 is raised, magazine 2782 may be rotated to select a head (for example chosen from amongst head 2719a and/or head 2719b and/or head 2719c). The selected head (for example head 2719c) may be positioned directly aligned with the loading aperture. Then magazine may be lowered (for example as illustrated in FIG. 27B) engaging head 2719c with an extender rod (for example extender rod 2684b). Head 2782 may then be disconnected from magazine 2782 and/or pushed distally into access tube 410.

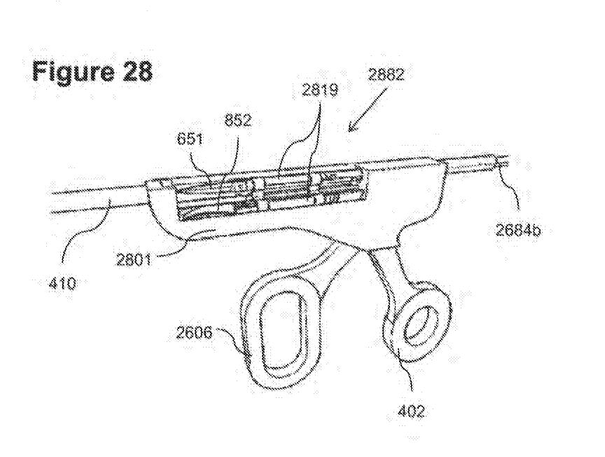

FIG. 28 illustrate an automatic magazine style handle for a laparoscopic access system in accordance with an embodiment of the present invention. Optionally a handle (for example in a tube connector 2801 portion thereof) may include a magazine 2882 optionally a set of heads 2819 lined up inside the magazine and biased to engage each one after a disengaging the previous head (for example like a magazine of an automatic rifle). Alternatively or additionally, magazine 2882 may include an internally mounted rotating set of heads 2819. As one head 2819 is removed, the next head is automatically engaged. In some embodiments, the magazine may be removable and a single tool shaft may be inserted (for example as illustrated in FIG. 4). Optionally the handle may include a switch to allow and/or inhibit complete removal of shaft 2684b.

It is expected that during the life of a patent maturing from this application many relevant technologies will be developed and the scope of the terms are intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references; unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within mat range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent is if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A laparoscopic tool system, comprising:
(a) a rigid tube adapted for insertion into a human body and long enough for laparoscopic use;
(b) a handle coupled to said tube; and
(c) a tool body attachable to a manual manipulator and including a tool head, slidably received in said rigid tube and long enough to extend from said manipulator with said tool head extending out a distal side of said rigid tube and wherein said tool body mechanically couples force from said manipulator to said tool head,
wherein said system, when said rigid tube is fully inserted into a body, rigidly extends less than 60% a length of said rigid tube out of said body.

2. A system according to claim 1, wherein said tool head is passable from a proximal opening to a distal opening of said rigid tube.

3. A system according to claim 1, wherein said tool body is slidable to extend at least half its length proximally from said handle.

4. A system according to claim 1, wherein said rigid tube is adapted to have a length of at least 15 cm inserted into a body.

5. A system according to claim 1, wherein said handle is adapted to extend at most 20 cm from a body, when said rigid tube is maximally inserted into a body.

6.-38. (canceled)

39. A method of laparoscopic surgery, comprising:
(a) inserting a tool head into the body at the end of a tool body coupled to a tool handle, to a location in the body;
(b) retracting the tool head and the tool body out of the body and at least partially out of said handle;
(c) advancing a different tool head into the body to said location; and
(d) manipulating said tool head using a manipulator coupled to said tool handle.

40. A method according to claim 39, wherein said inserting and advancing use a same rigid tube as a guide which reaches to said location.

41. A method according to claim 39, wherein said retracting comprises completely separating said tool body from said handle.

42. A method according to claim 39, wherein said retracting comprises separating said tool head from said tool body.

43. A method according to claim 39, comprising replacing said tool head by other than a physician who performs said manipulating.

44. A method according to claim 39, comprising initially inserting said tool body into an abdomen without a port.

45.-47. (canceled)

48. A cutting laparoscopic tool, comprising:
a tool body having a slot formed in a distal end portion thereof; and
a blade slidably received in said slot and mounted on a rigid elongate extender and having an outer diameter within 15% of an outer diameter of said tool body.

49. A tool according to claim 48, wherein said slot is formed in a distal end of a tool head forming a distal end of said tool body, said tool head configured to open and close.

50. A tool according to claim 48, wherein said slot is formed in a proximal end of a tool head forming a distal end of said tool body and said tool head does not allow said blade to extend past the tool head.

51. A tool according to claim 49, wherein said blade is configured for forming an abdominal incision for advancement of said tool body therethrough.

52. A tool according to claim 49, wherein said blade is configured for cutting tissue held by said tool head, within said tool head.

53.-57. (canceled)
